(19) United States
(12) Patent Application Publication
(10) Pub. No.: US 2001/0051714 A1
(43) Pub. Date: Dec. 13, 2001

(54) Linear probe carrier

(76) Inventors: Shiping Chen, Rockville, MD (US); Yuling Luo, Castro Valley, CA (US)

Correspondence Address: Charles D. Holland, Morrison & Foerster LLP, 755 Page Mill Road, Palo Alto, CA, 94304-0792, US

(21) Appl. No.: 09/758,873

(22) Filed: Jan. 10, 2001

Related U.S. Application Data
(60) Provisional application No. 60/175,225, filed on Jan. 10, 2000.
(60) Provisional application No. 60/190,495, filed on Mar. 20, 2000.
(60) Provisional application No. 60/227,874, filed on Aug. 25, 2000.
(60) Provisional application No. 60/244,418, filed on Oct. 30, 2000.

(51) Int. Cl. C12Q 1/68; C07H 21/04; C12M 1/34; G01N 33/543

(52) U.S. Cl. 536/24.3; 435/6; 435/287.2; 436/518

(57) ABSTRACT

The invention relates to a probe carrier in which a flexible substrate carries a one-dimensional configuration of probes wherein each different type of probe is attached to its own discrete portion of the substrate. The invention also relates to a probe carrier in which a flexible substrate such as a tape or fiber carries a two-dimensional configuration of probes. Furthermore, systems for fabricating and packaging flexible probe carrier threads are presented. Flexible probe carrier threads are packaged in forms of pins, rods, coils and spools to increase efficiency of hybridization and generate compact formats for transportation and use of probe carriers. Novel methods for hybridization of packaged probe carriers are disclosed. Methods for reading results of hybridization to packaged probe carriers are also disclosed.

### RELATED APPLICATIONS

This application claims the benefit of U.S. application Ser. No. 60/175,225, filed Jan. 10, 2000, 60/190,495, filed Mar. 20, 2000, 60/227,874, filed Aug. 25, 2000 and 60/244,418, filed Oct. 30, 2000. This application is also related to the PCT application entitled “Linear Probe Carrier,” Inventors Shiping Chen, Yuling Luo, and Anthony Chen, attorney docket number 473532000140, filed on even date herewith. Each of these applications is incorporated by reference herein in its entirety as if fully set forth below.

### TECHNICAL FIELD

This invention relates generally to the field of target analysis by binding to probes, as is commonly found in DNA sequence identification. This invention also relates to arrangements of immobilized nucleic acid probes on a solid substrate. More particularly, the invention relates to packaging of probe carrier threads wherein probes are immobilized in an array alone a flexible carrier.

### BACKGROUND OF THE INVENTION

Identification of molecular structure has become very important in research and in many industries, and the analysis of biological molecules such as nucleic acids and proteins forms the basis of clinical diagnostic assays. The procedures utilized often involve large numbers of repetitive steps which consume large amounts of time. (see, e.g., Sambrook, J., et al., Molecular Cloning: A Laboratory Manual. Cold Spring Harbor Laboratory Press, Cold Spring Harbor, N.Y. (2nd ed. 1989)). Simpler and quicker analysis of molecules has been provided by the development of arrays of test sites formed on a planar substrate. Each of the test sites includes probes which bind with samples applied to the device. Such probes may be oligonucleotides, proteins, antibodies, or cell-binding molecules and the choice of probes is theoretically limited only by the possibilities of specific binding to or reaction with sample. The binding of a sample to a probe is detected, and the probe identified, thereby identifying the sample. Technology has primarily developed around the use of these two-dimensional, planar arrays, especially in the area of arrays of oligonucleotides, which have become small and dense enough to be termed microarrays.

The ability to manufacture microarrays in an efficient and cost-effective manner is of considerable interest to researchers worldwide and of significant commercial value. The importance of the microarray technology to the biotechnology industry and to the entire health care sector cannot be overstated. A microarray is capable of dramatically boosting the efficiency of traditional biochemical experiments. Tests that would have taken years can now be completed in hours or even minutes. The applications of this technology affect more than the healthcare sector including gene profiling, disease diagnostics, drug discovery, forensics, agronomics, biowarfare and even biocomputers. Various types of microarray manufacturing devices and technologies have been described.

The current direction of technical development continues to be toward ever-denser two dimensional arrays of probes on rigid substrates. This approach presents a number of problems. First, as the number of test sites in an array is increased, the complexity of fabricating the array or pluralities of arrays is greatly increased. Second, the conventional methods of placing bio-molecules as probes on specific test sites—photolithography, mechanical spotting, and ink jetting—are time-consuming, expensive, often lack the desired accuracy and do not meet the desired size constraints. Photolithographic synthesis of probes in situ is a labor intensive technique that may not provide satisfactory accuracy and has a limited range of probe lengths. Mechanical spotting is a slow process in which the smallest test site size is limited by the nature of the process. Chemical ink jetting has an inaccuracy similar to in-situ synthesis and test site size limits similar to mechanical spotting. Third, because of the complexity and extreme precision required in manufacturing individual arrays, and the low throughput, the fabrication cost of each array is very high, often thousands of dollars for arrays containing enough probes to evaluate complex biological samples. Fourth, the expense and complexity of the reading devices for detecting probe-sample hybrids, which is already extremely high, increases with each increase in array density, and because the reader has to carry out a two-dimensional scan with a very high spatial precision (in the order of 10 μm), processing time for each scan also increases with increasing density of the two-dimensional probe array.

In addition, the basic operating principle of microarray involves a probe immobilized on a substrate to react with specific molecules in sample fluid. Hybridization requires providing probes with sufficient chances to meet their complementary molecules. In existing systems, this is achieved through diffusion or driving the sample fluid across the microarray. The former is a random process and the later requires complex microfluidic systems.

Hence, there is a need for an easily- and rapidly-constructed, inexpensive probe carrier which can accommodate thousands or hundreds of thousands of probes, which is capable of compact storage and use, can be manufactured at a high rate of throughput, can facilitate probe/target interaction with a high efficiency and does not require expensive and highly precise reading devices, can carry detailed information about individual probes or groups of probes on the substrate along with the probes themselves, can accommodate probes of varying lengths and degrees of complexity in customized groups, and which is compact, easy to use, and inexpensive enough to allow one-time use with resulting high accuracy.

There is also a need for improved packaging of such probe carriers whereby the required amount of hybridization fluids is minimized and large numbers of probes can be immobilized on a substrate without the concomitant increase in size as a standard two-dimensional gene chip matrix would necessitate.

### BRIEF SUMMARY OF ASPECTS OF THE INVENTION

The present invention provides a new direction and approach in making a probe carrier or probe configuration that does not require dense two-dimensional symmetrical arrays built upon a rigid substrate and also does not inherently limit the size of the probes that can be attached to a substrate. In addition, the present invention can be relatively easily fabricated through use of assembly-line-like techniques.

The invention provides a probe carrier in which a plurality of probes are immobilized in discrete areas, one probe per area, on an elongated flexible substrate with a length:width ratio of at least about 5:1, at least 50:1, at least 500:1, at least 10,000:1, or at least 100,000:1. In one embodiment, the length of each probe-containing area does not exceed 1000 micrometers, in another embodiment the length of each probe-containing area does not exceed 500 micrometers, in another embodiment the length of each probe-containing area does not exceed 100 micrometers, in still another embodiment the length of each probe-containing area does not exceed 50 micrometers, and in yet a further embodiment the length of each probe-containing area does not exceed 20 micrometers.

The invention also provides a probe carrier in which a plurality of probes are immobilized in discrete areas, one probe per area, on a flexible substrate with a length:width ratio of at least about 5:1, where the substrate has layer on its surface, and where the probes are immobilized on the surface of the layer. In a further aspect, the invention also has a second layer between the first layer and the substrate. In one embodiment, the first layer comprises silica and the second layer comprises a metallic material.

The invention also provides a linear one-dimensional arrangement of probes immobilized in a single file on the surface of a flexible substrate, in which the linear density of the probes exceeds 10 probes per linear cm, or preferably 50 probes per linear cm. In another aspect of the invention, the linear density of the probes exceeds 100 probes per linear cm, in a further aspect of the invention, the linear density of the probes exceeds 200 probes per linear cm, and in yet a further aspect of the invention, the linear density of the probes exceeds 500 probes per linear cm.

In addition, the invention provides a plurality of probes immobilized on discrete areas of the surface of a flexible tape substrate, one probe per area, where the tape has a thickness not exceeding 500 micrometers. In another aspect of the invention, the tape does not exceed 100 micrometers in thickness, and in yet another aspect the tape does not exceed 20 micrometers in thickness.

The invention also provides a plurality of probes immobilized on discrete areas of the surface of a flexible fiber substrate, one probe per area, where the fiber has a diameter not exceeding 500 micrometers. In another aspect of the invention, the fiber does not exceed 200 micrometers in diameter, in yet another aspect the fiber does not exceed 100 micrometers in diameter, and in still another aspect the fiber does not exceed 20 micrometers in diameter.

All of the above aspects of the invention may further include a first marker which conveys information about a first set of probes, and a second marker which conveys information about a second set of probes. In some embodiments, the markers may be optical markers, such as optical bar codes or fluorescent markers, in another embodiment the markers may be magnetic. In one embodiment which includes markers, the probes are polynucleotides, in another embodiment which includes markers, the probes are polypeptides, in yet another embodiment which includes markers, the probes are antibodies, and in still another embodiment which includes markers, the probes are selected from the group consisting of cell surface receptors, oligosaccharides, polysaccharides, and lipids.

Also in all of the above embodiments, whether or not they include markers, the apparatus also includes a first layer, with the probes immobilized on that layer; the layer may be composed of silica. Alternatively, the apparatus includes both a first layer and a second layer; the first layer may be silica and the second layer may be a metallic material. If the second layer is metallic, it may also be magnetizable.

In all of the above embodiments the probes may be arranged as a linear configuration of spots, or as a linear configuration of stripes with the stripes being at an angle to the long axis of the substrate.

Also, in all the above embodiments, the probes may be polynucleotides, or polypeptides, or antibodies, or ligands, or be selected from the group consisting of cell surface receptors, oligosaccharides, polysaccharides, and lipids. If the probes are polynucleotides, they may be DNA, and if DNA, they may be single-stranded DNA.

The substrate for the invention may be silica glass, or plastic, or a metallic material, or a polymer, and if a polymer, the polymer may be selected from the group consisting of polyimide and polytetrafluoroethylene. A preferred substrate is an optical fiber. If the substrate is a metallic material, it may also have a layer between the substrate and the probes, so that the probes are immobilized on the layer; the lay may be silica. Furthermore, the metallic substrate may be magnetizable.

The invention may be wound about a drum or a plurality of drums. It may also be wound upon itself in a flat spiral, with or without a flat backing, and it may be further attached to a spool at the center of the flat spiral. In addition, the outermost end of the substrate in the spiral may be extended and attached to a second spool.

In a different aspect, the invention comprises an apparatus for transporting a plurality of probe fluids to a substrate to print a probe array. Typically, the apparatus includes a reservoir with a plurality of wells, and a set of capillaries, where the capillaries are arranged so that one end of each capillary is connected to a well in the reservoir, such that the contents of the well may enter the capillary, and the second end of the capillaries are arranged in a flat single file row. In one embodiment of this apparatus, the reservoirs are wells in a microtiter plate. When using the apparatus of the invention, probe fluids may be moved from the wells in the reservoir into the capillary tubing by applying a pressure differential between the reservoir and the tubing, and/or by providing a voltage between the reservoir and the substrate. The capillary may be positioned parallel to and may move across the longitudinal axis of the elongated probe substrate to deposit a set of probes on the substrate. Other methods of probe deposition are described below in further detail.

A second probe transport apparatus has a row of probe containers configured in a fashion similar to a conveyer belt. The row of containers is moved at one speed and direction to intersect with the substrate, which is moving at another speed and direction, to deposit probes, one by one, onto the substrate. In an alternative configuration, the row of probe containers is moved to intersect a moving row of spotters, which are made of a flexible strand of material, such that each spotter intersects a container to transfer probe from the container to the spotter. A conveyor, for instance, also moves a substrate so that it intersects the row of probe-carrying spotters such that each spotter deposits its probe onto the substrate after it has picked up the probe from a container. The row of spotters may be configured as a loop, and further the spotters may be washed in a washing station after they have deposited probe on the substrate and before they return to the containers.

The invention also includes methods of depositing probes from a fluid transportation apparatus onto the substrate surface. In one method, probes are painted as strips on the substrate. The probe may be carried on a thin, flexible and elastic spotter, which contacts the substrate surface in a brushing action to paint the strip. In one embodiment, the spotter can be a silica capillary or fiber. Alternatively, the capillary or fiber can be made of other materials such as metal, ceramics, polymer, or other material that is capable of transporting the probe-containing fluid. In other methods, the probes may be deposited in a non-contact fashion either as strips or dots. These methods include magnetic, electric, thermal, acoustic and inkjet deposition. In a magnetic deposition method, the probe is attached to magnetic beads. An electromagnet placed underneath the substrate is activated as the spotter carrying a probe immobilized on magnetic beads intersects the substrate. The magnetic field generated by the electromagnet pulls the probe from the fluid transportation apparatus to deposit the probe onto the surface of the substrate. In an electric deposition method, a voltage of appropriate polarity is applied between the substrate and the delivery device to establish an electric field to push the electrically charged probes (such as oligonucleotides) onto the substrate surface. In a thermal deposition method, rapid, localized heating is introduced into the path of fluid, producing a rapid local volume expansion (a bubble) that propels probe fluid onto substrate. Rapid heating can be introduced either electrically by a resistance heating wire or optically using suitable laser light. In acoustic deposition, an ultrasonic pulse is introduced into probe fluid which propels a droplet out onto the substrate surface. In inkjet deposition, a piezoelectric actuator is built in the probe fluid container. Activated by a voltage signal, the piezoelectric actuator rapidly reduces the volume of the container thus pushing out the probe fluid onto the substrate surface.

In another alternative method, painting the probes in strips on the substrate may be accomplished by a probe deposition apparatus in which a matrix of fibers is dipped into a corresponding matrix of reservoirs, each reservoir containing probe, then the matrix of fibers is moved across a first section of substrate, with the fibers and the substrate positioned so that each fiber deposits a separate line of probe across the substrate with the desired substrate between lines. In this method, the fiber matrix may be washed and dipped into another matrix of reservoirs then moved across another section of substrate to deposit another set of strips of probes.

In all of the above methods, the substrate may be a plurality of fibers arranged in parallel, so that several fibers receive probe with one pass of the probe-deposition instrument, or the substrate may be a tape, where, after probe deposition, the tape is optically cut along its long axis to produce a plurality of probe-carrying tapes.

In all of the above methods, the probes may be covalently linked to the substrate.

In all of the above methods, the further step of adding markers to the substrate may be included.

Among other factors, the invention is based in the technical finding that a probe carrier having a one-dimensional configuration of probes on a flexible substrate provides a simple, economical, reliable, and classification-specific way to identify the presence of target molecules in a sample. Further, probes on a probe carrier of this invention are not limited in size. These technical findings and advantages and others are apparent from the discussion herein.

This invention encompasses a new way of improving the efficiency of hybridization to target or reaction with target. This method involves moving the probe carrier through sample fluid to enhance the chance for the probes immobilized on the carrier to mix with their target molecules in the sample fluid. The carrier may take a variety of forms including thread, tape, slide, coil, drum or pin. The movement can involve translation, rotation or vibration of the probe carrier, either alone or in combination.

This invention also includes several designs of hybridization device, where the probe carrier is inserted into a chamber containing the sample fluid. The gap between the probe carrier and the inner wall of the chamber is minimized to reduce the volume of sample fluid. The probe carrier is driven to move within the chamber to improve the efficiency of probe/target interaction. An additional method of hybridization enhancement involves applying a voltage between the probe carrier and the wall of the hybridization chamber. At one polarity, the electric field pulls target molecules towards the probes on the carrier, which increases the local concentration of target molecules and improves the likelihood of hybridization. At the opposite polarity, the electric field repels target molecules away from the probes, which can help to increase the specificity of hybridization. By alternating the polarities at suitable frequencies, the hybridization efficiency between the target and probe can be improved.

The invention may be used in the analysis of known point mutations, expression analysis, genomic fingerprinting, polymorphism analysis, linkage analysis, characterization of mRNAs and mRNA populations, sequence determination, sequence confirmation, disease diagnosis, and other uses which will be apparent to those of skill in the art.

### BRIEF DESCRIPTION OF THE DRAWINGS

FIG. 1 illustrates one embodiment of the probe carrier, in which probes are immobilized as spots on a substrate, which also carries markers in the form of optical bar codes. Probes may also be immobilized as stripes or as rows of spots, and markers may be optical, magnetic, or any other identifiable marking;

FIG. 2a is a cross-sectional view of a probe-carrier, in which the probes are immobilized in a notch in the carrier; and FIG. 2b is a cross-sectional view of two layers of a probe-carrier in which the probes are immobilized in a notch on the carrier, and illustrates how the position of the immobilized probes in the notch protects them from friction with the next layer.

FIG. 3 illustrates an apparatus and method of fabricating probe carriers in which a plurality of tubes transports probes from reservoirs to the substrate and “paints” the probes in stripes on the substrate.

FIG. 4 schematically represents a method of fabricating probe carriers in which individual probe containers pass across a substrate or plurality of substrates and deposit probe on the substrate as each probe container passes over the substrate.

FIG. 5 illustrates various types of probe containers which may be used in the preceding fabrication technique, and the means that each employs to deposit probe from the carrier onto the substrate.

FIGS. 6a-6e illustrate methods of fabricating probe carriers. FIG. 6a illustrates a method of fabricating probe carriers in which probe is contained in liquid in individual reservoirs. FIG. 6b illustrates a method of fabricating probe carriers in which a moving belt of spotters intersects the reservoirs so that each spotter picks up a separate probe. FIG. 6c illustrates a method of fabricating probe carriers in which the moving belt of spotters, with probe associated, intersects a substrate or plurality of substrates and deposits the probe thereon. FIG. 6d illustrates a method of fabricating probe carriers in which the spotters can be arranged in a continuous loop in which individual spotters are washed and reused for spotting new probes which are provided from a reservoir array. FIG. 6e depicts a method of transferring probe from a reservoir to a spotter. In this configuration, the spotter moves under the substrate and the substrate surface is positioned face down to allow the spotter to deposit the probe from underneath the substrate.

FIG. 7a and 7b illustrate another method of constructing probe carriers, in which a matrix of spotters dips into a corresponding matrix of wells, each well of which contains a probe, then the spotter matrix is brushed across a substrate or plurality of substrates at such an angle that each spotter deposits a separate line, then the spotter array is washed and moves to a new matrix of probe containing wells, and repeats the dipping-brushing-washing cycle on a new section of substrate.

FIG. 8 illustrates configurations of a probe carrier pin and a probe carrier rod.

FIG. 9 illustrates fabrication methods for a probe carrier pin and a probe carrier rod.

FIG. 10a illustrates a top view of a flexible probe carrier in a coil configuration. FIG. 10b illustrates a side view of a flexible probe carrier in a coil configuration. FIG. 10c is a cross-sectional view of two adjacent turns of a probe-carrier thread in which the probes are immobilized within notches on the carrier.

FIG. 11a illustrates a flexible probe carrier in a spool configuration packaged in a mini cassette. FIG. 11b is a cross-sectional view of two layers of a probe-carrier in the spool in which the probes are immobilized in a notch on the carrier, and illustrates how the position of the immobilized probes in the notch protects them from friction with the next layer.

FIG. 12 illustrates a method of using an electric field to control hybridization to a probe carrier.

FIG. 13 illustrates a method of hybridization to a probe carrier pin.

FIG. 14 illustrates a method of parallel hybridization of multiple target samples in standard microtiter plate format using probe carrier pins.

FIG. 15 is a view of hybridization equipment for a probe carrier rod as viewed along the axis of the rod.

FIG. 16 is a side view of hybridization equipment for a probe carrier coil.

FIGS. 17a and 17b illustrate hybridization equipment for a probe carrier spool.

FIG. 18 illustrates a reader for scanning a probe carrier pin or a probe carrier rod.

FIG. 19 illustrates a reader for scanning a probe carrier coil.

FIG. 20 illustrates a reader for scanning a probe carrier spool.

### DETAILED DESCRIPTION OF THE INVENTION

1. The Probe Carrier Apparatus

A. General description

Scanning and imaging of microarrays can be facilitated by one-dimensional arrays of probes because such arrays do not require the high degree of precision necessary for imaging in two dimensions. A number of apparatuses which utilize polynucleotides bound to optical fibers may be found in the following: “Nucleic Acid Biosensor Diagnostics,” Krull, et al., WO #98/58079 and WO #95/26416; “Fiber optic biosensor for selectively detecting oligonucleotide species in a mixed fluid sample,” Walt et al., WO #98/50782; “Analytical method for detecting and measuring specifically sequenced nucleic acid,” Sutherland, et al., EP #0245206; “Gene probe biosensor method,” Squirrel, WO #93/06241; “Nucleic acid assay method,” Hirschfield, U.S. Pat. No. 5,242,797; Piunno et al., Fiber-optic DNA sensor for fluorometric nucleic acid determination, Anal. Chem. 67:2635-2643, 1995; Uddin et al, A fiber optic biosensor for fluorimetric detection of triple-helical DNA, Nucleic Acids Res. 25:4139-4146, 1997; Abel et al., Fiber-optic evanescent wave biosensor for the detection of oligonucleotides, Anal. Chem. 68: 2905-2912, 1996; Kleinjung et al, Fibre-optic genosensor for specific determination of femtomolar DNA oligomers, Anal. Chim. Acta 150:51-58, 1997; Zhang et al., A chemilluminescence fiber-optic biosensor for detection of DNA hybridization, Anal. Lett. 32:2725-2736, 1999; Ferguson et al., A fiber-optic DNA biosensor microarray for the analysis of gene expression, Nature Biotech., 14:1681-1684, 1996.

However, these apparatuses typically involve attachment of only one probe molecule sequence on the glass surface of single optical fibers. Krull, et al. (WO #98/58079) have theorized the use of an undifferentiated mixture of more than one type of probe, however, the number of different probe sequences is sharply limited in these techniques by the unorganized distribution of probe molecules, which necessitates that each individual probe molecule be tagged by, for example, fluorescent labels (as suggested by Krull et al.), in order to identify it and distinguish it from its local neighbors, which may be probes with different sequences. In addition, previous approaches have used only short sections of fiber, on the order of a few centimeters or less, limiting the number and kinds of probes that can be immobilized. Finally, the previous techniques utilize the optical fiber on which probes are immobilized to conduct light both to and from the markers of hybridization, which are typically fluorophores. This detection technique relies on evanescent illumination from the optical fiber, which is inherently limited to the area immediately adjacent to the fiber surface, does not provide discrimination among groups of probes, and is limited in sensitivity. Furthermore, the use of the optical fiber itself to conduct the excitation and emission light limits one to the use of optical fibers alone as substrates on which to immobilize probes and precludes the use of other substrates, such as metal wire or polymer, which may offer other advantages such as the ability to carry information about individual probes or groups of probes, as well as advantages in hybridization.

Unlike the established “gene-chip” technology where DNA probes form a two dimensional matrix of spots on a planar slide, a “probe carrier thread” immobilizes the probes in an one dimensional array along a single length of thin, flexible thread. A probe carrier thread system is comprised of three essential elements: probe carrier thread configuration and fabrication, hybridization and readout. Improved packaging of a probe carrier thread by use of probe carrier pin, probe carrier rod, probe carrier coil and probe carrier spool technologies increases the density of probes and enhances the inherent advantages of the probe carrier thread technology. “Flexible,” as used herein, means capable of being bent, wound, coiled or otherwise flexed to the degree necessary for the operation of the invention without breaking.

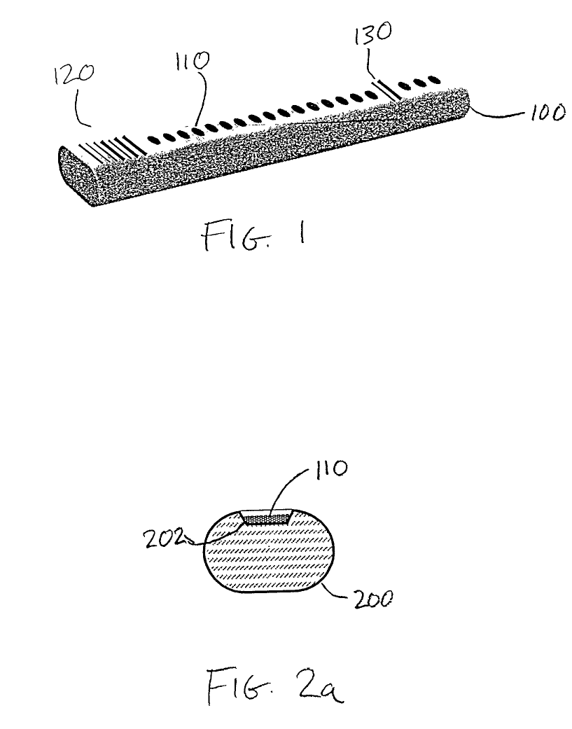

As illustrated in FIG. 1, in one embodiment of the invention probes are immobilized as spots (110) at the center or as narrow stripes (see FIG. 4, 404) across the width of a long, thin and flexible substrate (100). Alternatively, probes can be immobilized as successive rows of spots, said rows being at an angle to the long axis of the substrate. The length:width ratio of the substrate is at least about 5:1, preferably at least 50:1, more preferably at least 500:1, and most preferably at least 10,000:1. The length:width ratio of the probe-containing portion of the substrate is at least about 5:1, preferably at least 50:1, more preferably at least 500:1, and most preferably at least 10,000:1. The length:width ratios, the flexibility of the substrate, and the positioning of the probes in a one-dimensional or nearly one-dimensional arrangement, allow for new and simplified methods of manufacturing, using, and analyzing the probe carrier.

A “probe,” as used herein, is a set of copies of one type of molecule or one type of molecular structure which is capable of specific binding to or specific reaction with a particular sample or portion of a sample. The set may contain any number of copies of the molecule or multimolecular structure. “Probes,” as used herein, refers to more than one such set of molecules or multimolecular structures. The molecules or multimolecular structures may be polynucleotides, polypeptides, oligosaccharides, polysaccharides, antibodies, cell receptors, ligands, lipids, cells, small molecules as are used to e.g. screen drugs as are used in screening pharmaceuticals, or combinations of these structures, or any other structures to which samples of interest or portions of samples of interest will bind or react with specificity. The probes may be immobilized on the substrate by either covalent or noncovalent attachment. “Flexible,” as used herein, means capable of being bent, wound, coiled or otherwise flexed to the degree necessary for the operation of the invention without breaking. “Width” of the substrate is defined as the length of the longest perpendicular to the long axis of the substrate which is entirely contained within the substrate. “Width” of the probe-containing portion of a cylindrical such as a thread substrate is defined as the linear distance of the longest arc, contained within the probe-containing portion of the substrate, which is perpendicular to the long axis of the probe-containing portion of the substrate. Length of the probe-containing portion of the substrate is the linear distance along the long axis of the substrate from the first probe to the last probe of the probes on the substrate or, if there is a substantially larger gap between probes that form groups of probes, the length is the linear distance from the first to last probe in the group.

As with conventional two-dimensional microarrays arranged on a planar surface, the present apparatus is used to analyze samples by 1) distinguishing the probes which have bound or react with sample or sample fragment from those that have not bound sample, then 2) establishing the identity of the probe(s) which have bound sample.

A further way to identify probes is by markers which serve to identify individual probes or sets of probes. Such markers may be used to convey more information than simply the identity of the probe.

The long, thin, and flexible nature of the probe carrier lends itself to numerous novel means of containment and use. The probe carrier may be packaged in a number of formats including but not limited to a pin, a rod, a coil and a spool. Hybridization methods are considerably enhanced by requiring less hybridization fluid and enhanced mixing. A flexible probe carrier packaged in a spool is especially advantageous in applications that require high volume, low to medium scale microarrays, such as those involved in disease diagnostics and management in major hospitals. In these applications, the required number of probes in the array is small (in the range of several hundreds to several thousands) but a very large number of the same type of arrays may be consumed every day. With the flexible probe carrier format, tens thousands of copies of identical sets of probes are arrayed repetitively along a continuous length of thread and sealed in a large coil or spool. A filly automated system integrates equipment for every stage of the analytical process, such as a hybridization station and a scanner. The machine takes in patients' DNA samples and feed the flexible probe carrier through the entire process and produces analysis and results in a fully automated manner without human intervention.

B. Specific Description

1.1 Substrate.

The substrate of the invention can be made of various materials. The requirements of the substrate are that it have sufficient flexibility to withstand the conformational changes necessary to the manufacture and use of the apparatus, and that it be capable of immobilizing the particular probes to be used, or be capable of modification (for example, by coating) so that it is capable of such immobilization. The substrate may also comprise various layers made of different materials, each of which has a function in the apparatus.

Specific embodiments will require differing degrees of flexibility. Flexibility may be measured by the ability to withstand winding to a certain diameter, for example a diameter of 10 cm, 5 cm, 2 cm, 1 cm, 0.5. cm, or 0.1 cm. Preferred materials for the substrate of the present invention are silica glass, metallic materials, plastics, and polymers of sufficient strength to withstand the processes of manufacture and use.

For immobilizing polynucleotides and polypeptides, silica, i.e. pure glass, is a preferred material because polynucleotides and polypeptides can be covalently attached to a treated glass surface and silica gives out a minimum fluorescent noise signal. The silica may be a layer on another material, or it may be the substrate, core or base material of the apparatus, or both. One embodiment of the present invention comprises a metal wire as the core substrate, with a coating of silica on it for probe immobilization. Another embodiment comprises a plastic or polymer tape as a base substrate, with a coating of silica for probe embodiment. In this embodiment, a further layer of metallic material may be added, either on the opposite side of the tape from the silica layer, or sandwiched between the silica layer and the polymer or plastic. Yet another embodiment of the invention is a silica fiber with a layer of metallic material on the silica core and another layer of silica on the metallic material; probes are immobilized on this outer silica layer.

Optical fibers. The probe carrier thread can be made of different materials. A preferred material is silica because DNA can be covalently attached onto a treated glass surface and silica emits minimum fluorescent noise. Contrary to common perception that glass is a rigid and easily breakable material, fibers made of silica are flexible and have great elastic strength. For example, the optical fiber currently mass-produced for the telecommunication industry is made of silica. Optical fiber is a substrate material which is made of primarily of silica and provides the necessary requirements. Although such fibers are manufactured for the purpose of transmitting light, the present invention does not require this feature of the fibers (although it may be used in some embodiments). Rather, it is other features of the optical fiber which make it particularly advantageous for the present invention. The mechanical strength of optical fibers has been measured at 7 GPa, about 4 times that of the strongest steel while only ⅙ of its weight. Optical fibers are also highly flexible. Standard 125 μm diameter fibers can be coiled in loops down to 5 mm in diameter without breakage.

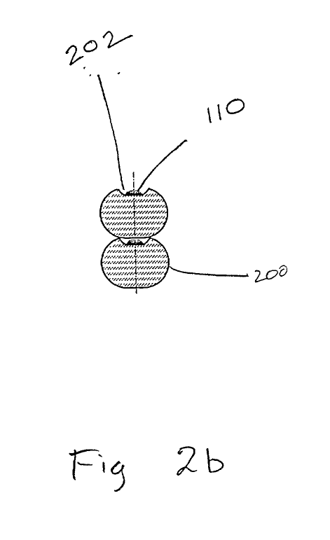

Also, the process of making optical fibers lends itself to customization, especially in terms of the cross-sectional shape of the fiber. Optical fibers are made from preforms, typically 1 meter long and 3 cm in diameter, fabricated using silica. The center portion of the preform is doped with Germanium to create a core with higher refractive index to guide light through. Then the perform is installed on a fiber draw tower in clean room enviromnent, which heats it to the melting point and pulls out the fiber on to a large drum. The cross-section shape of the fiber generally resembles that of the preform and the diameter of the fiber can be controlled through the pulling speed. Most optical fibers on the market have circular cross-sections and an outer diameter of 125 μm. However, other diameters and shapes, particularly “D” cross-sectional shapes are also available. This is especially useful if the final probe carrier is to be wound upon itself for storage and ease of use. As shown in FIG. 2a, the cross-section of the fiber can be adjusted using a notched, D-shaped preform so that the fiber (200) has a notch, or groove (202) , in which probes (110) are immobilized. This design protects the probes of one layer from friction with the substrate of a succeeding layer, as shown in FIG. 2b, where the cross-sections of two successive layers are shown one on top of the other.

In addition, owing to the high purity of the material and careful control of the fabrication process, optical fibers have very few structural defects. Also, optical fibers have excellent dimensional precision. Diameters are controlled to within ±1 μm. Finally, the cost of optical fibers is very low, at about 1˜2¢ per meter. This is because the fabrication process of fibers is fairly straightforward and a single preform can produce up to 100 km of standard telecommunication fibers.

A number of apparatuses which utilize polynucleotides bound to optical fibers have been described in the following: “Nucleic Acid Biosensor Diagnostics,” Krull, et al., WO #98/58079 and WO #95/26416; “Fiber optic biosensor for selectively detecting oligonucleotide species in a mixed fluid sample,” Walt et al., WO #98/50782; “Analytical method for detecting and measuring specifically sequenced nucleic acid,” Sutherland, et al., EP #0245206; “Gene probe biosensor method,” Squirrel, WO #93/06241; “Nucleic acid assay method,” Hirschfield, U.S. 5,242,797; Piunno et al., Fiber-optic DNA sensor for fluorometric nucleic acid determination, Anal. Chem. 67:2635-2643, 1995; Uddin et al, A fiber optic biosensor for fluorimetric detection of triple-helical DNA, Nucleic Acids Res. 25:4139-4146, 1997; Abel et al., Fiber-optic evanescent wave biosensor for the detection of oligonucleotides, Anal. Chem. 68: 2905-2912, 1996; Kleinjung et al, Fiber-optic genosensor for specific determination of femtomolar DNA oligomers, Anal. Chem. Acta 150:51-58, 1997; Zhang et al., A chemilluminescence fiber-optic biosensor for detection of DNA hybridization, Anal. Lett. 32:2725-2736, 1999; Ferguson et al., A fiber-optic DNA biosensor microarray for the analysis of gene expression, Nature Biotech., 14:1681-1684, 1996.

These apparatuses typically involve attachment of only one probe molecule sequence on the glass surface of single optical fibers, greatly limiting their usefulness. Previous approaches have used only short sections of fiber, on the order of a few centimeters or less, limiting the number and kinds of probes that can be immobilized. Finally, the previous techniques utilize the optical fiber on which probes are immobilized to conduct light both to and from the markers of hybridization, which are typically fluorophores. This detection technique relies on evanescent illumination from the optical fiber, which is inherently limited to the area immediately adjacent to the fiber surface, does not provide discrimination among groups of probes, and is limited in sensitivity. Furthermore, the use of the optical fiber itself to conduct the excitation and emission light limits one to the use of optical fibers as substrates on which to immobilize probes and precludes the use of other substrates, such as metal wire or polymer, which may offer other advantages such as the ability to carry information about individual probes or groups of probes, as well as advantages in hybridization, as discussed below.

Commercial telecom fibers are coated with a layer of non-porous polymer, which is not optimal for probe immobilization. The coating can be removed by techniques known in the art, such as those described in U.S. Pat. No. 5,948,202, which is incorporated by reference herein in its entirety. However, bare fiber without this coating is prone to attack by water vapor, which generates micro-cracks on the fiber surface and degrades its strength. As a result, the bare silica fiber may not survive the very tough environment during the hybridization stage. There are several approaches to solving this problem.

One approach is to wind the fiber into a spiral coil along an elongated cylinder or drum after probe immobilization. The fibers sit side-by-side on the drum and are attached to its solid surface. The probes are aligned along a side of the fiber that is distal to the side of the fiber attached to the drum. The drum provides mechanical support to the fiber during hybridization of sample and detection of the hybridization pattern.

A second approach is to strengthen the fiber substrate by applying one or several layers of coating to the silica fiber, which protect the fiber from the onslaught of water vapor and at the same time maintain good binding to probes. The strengthened fiber can then be wound, for example, on a specially designed spool and assembled in a sealed cassette for transportation and handling. An example of a substrate strengthening method is to coat the fiber with a metallic material, then an additional layer of silica. To protect the bare silica fiber from moisture absorption, one or several layers of hermetic coating can be applied. Suitable coating materials including gold, silver and titanium due to their relative inertness in chemical solutions. Carbon coating is also widely used in the fiber optic telecommunications industry for hermetic sealing. This invention in one embodiment further provides an additional layer of silica coating over the hermetic layer(s) to provide covalent binding with DNA probes. Such a coating can be implemented through low cost sol-gel process and provides a surface for immobilization of probes, especially by covalent binding.

In addition to silica, other materials can also be used as the main body of the substrate. These include thin metal wires or strong polymer (polyimide or polytetrafluoroethylene (PTFE), for example) tapes. Again a sol-gel silica coating can be applied to the substrate to facilitate probe binding. For the polymer tape substrate, one can add a layer of metallic material sandwiched between the tape and the silica.

The metallic material element in all the substrate designs described above not only protects and/or strengthens the substrate, it may provide additional benefits during fabrication of the probe carrier and during binding of samples which carry a charge, which are described below.

The substrate is elongated. “Elongated,” as used herein, means that the length:width ratio of the substrate exceeds about 5:1, preferably exceeds 100:1, more preferably exceeds 1000:1, and most preferably exceeds 10,000:1. It is contemplated that the length: width ratio can be even greater, such as at least 100,000:1 or at least 1,000,000:1. As defined above, “width” of the substrate is defined as the length of the longest perpendicular to the long axis of the substrate which is entirely contained within the substrate. If the substrate is of varying widths, the width to be used to calculate the length:width ratio is the longest width. “Width” of the probe-containing portion of the substrate is defined as the longest arc (for an arc shaped probe-containing area, as is typically found on a cylindrical thread-like substrate) or the large lineal distance for a flat substrate, contained within the probe-containing portion of the substrate, which is perpendicular to the long axis of the probe-containing portion of the substrate. “Length” of the substrate is defined as the length of the long axis of the substrate. If the substrate has more than one length, the shortest of the lengths is used to calculate the length: width ratio.

The cross-section of the substrate can be of any shape. The “cross-section,” as used herein, is defined as the planar section through the substrate perpendicular to the long axis of the substrate. Although the cross-section can be any shape, two particular shapes represent different embodiments of the invention. First, as used herein, a “tape” refers to an embodiment utilizing a tape-, ribbon-, or strip-like substrate, whose cross-section is rectangular or nearly rectangular, or in the shape of a parallelogram. Such a tape will have a thickness, corresponding to the width of the cross-sectional area. In various embodiments of the invention, this thickness does not exceed 500 micrometers, or 100 micrometers, or 50 micrometers, or 20 micrometers. Second, as used herein, a “fiber” is an embodiment which utilizes a fiber-, thread-, or wire-like substrate, whose cross-section is rounded. The cross-section may be circular, elliptical, or partially circular, for instance as with a fiber with a D-shaped cross-section. The cross-section has a diameter, defined herein as the longest linear dimension of the cross section. In various embodiments of the invention, the diameter of the fiber does not exceed 500 micrometers, or 200 micrometers, or 100 micrometers, or 50 micrometers, or 20 micrometers. The terms “tape” and “fiber” are intended to represent two parts of a spectrum of cross-sectional shapes. The invention can, however, have a cross-section of any shape. The substrate may incorporate a groove or grooves running approximately parallel to the long axis of the fiber, in which probes are immobilized, as illustrated in FIG. 2a. Such a groove can be seen as an indentation in the cross-section. The use of such a groove or indentation reduces or eliminates friction between probes immobilized in the groove and other surfaces; for example, when the substrate is wound on itself in a spiral, probes immobilized on one winding would be protected from the substrate on the next winding due to being recessed in the groove. A D-shaped cross section incorporating such an indentation facilitates stacking of one layer of a winding on the next, as well as protection of probes. Other embodiments may utilize different cross-sections, which will be useful in the use of the apparatus, and will be apparent to one of skill in the art.

1.2 Probes

A “probe,” as used herein, is a set of copies of one type of molecule or one type of multimolecular structure which is capable of specific binding to a particular sample or portion of a sample. “Probes,” as used herein, refers to more than one such set of molecules. A probe may be immobilized on the substrate by either covalent or noncovalent attachment. Probes may be polynucleotides, polypeptides, oligosaccharides, polysaccharides, antibodies, cell receptors, ligands, lipids, cells, or combinations of these structures, or any other structures to which samples of interest or portions of samples of interest will bind with specificity. The set of probes chosen depends on the use of the apparatus. For example, if the apparatus uses polynucleotides as probes, if one is performing sequence analysis, one would prefer a complete or nearly complete set of n-mers; the use of such sets is more fully described in U.S. Pat. Nos. 5,700,637 and 6,054,270, which are hereby incorporated herein by reference in their entirety. On the other hand, if a device is to be used to analyze mutations or polymorphisms in a gene or set of genes, polynucleotides representing a complete or chosen set of mutations, such as substitution, deletion, and insertion mutations, for sections of the particular gene or genes of interest may be preferred. As a further example, in diagnostics such as for cancer-related mutations, particular mutational “hot spots” in a set of genes known to be associated with a particular cancer or cancers would be the areas to which complementary polynucleotides would serve as the set of probes. These examples are merely illustrative of the various custom sets of probes that might be selected for a particular apparatus and focus on polynucleotides because these are the types of probes now most commonly in use; it is to be understood that other types of probes and other sets of polynucleotides will be readily apparent to the skilled worker in the field.

As used herein, “polynucleotide” means a polymeric form of nucleotides of any length, which contain deoxyribonucleotides, ribonucleotides, and/or their analogs. The terms “polynucleotide” and “nucleotide” as used herein are used interchangeably. Polynucleotides may have any three-dimensional structure, and may perform any function, known or unknown. The term “polynucleotide” includes double- or single-stranded, and triple-helical molecules. Unless otherwise specified or required, any embodiment of the invention described herein that includes a polynucleotide encompasses both the double-stranded form and each of two complementary single-stranded forms known or predicted to make up the double stranded form. Relatively shorter lengths of polynucleotides (less than about 100 nucleotides) are also referred to as oligonucleotides.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. Analogs of purines and pyrimidines are known in the art, and include, but are not limited to, aziridinylcytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudo-uracil, 5-pentynyl-uracil and 2,6-diaminopurine. The use of uracil as a substitute for thymine in a deoxyribonucleic acid is also considered an analogous form of pyrimidine.

If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications included in this definition are, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s).

Further, any of the hydroxyl groups ordinarily present in the sugars may be replaced by phosphonate groups, phosphate groups, protected by standard protecting groups, or activated to prepare additional linkages to additional nucleotides or to solid supports. The 5′ and 3′ terminal OH groups can be phosphorylated or substituted with amines or organic capping groups moieties of from 1 to 20 carbon atoms. Other hydroxyls may also be derivatized to standard protecting groups.

Polynucleotides can also contain analogous forms of ribose or deoxyribose sugars that are generally known in the art, including, but not limited to, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. As noted above, one or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing and ether (—O—) linkage, aryl, alkenyl, cycloalkyl, cycloalkenyl or araldyl. Not all linkages in a polynucleotide need be identical. Substitution of analogous forms of sugars, purines and pyrimidines can be advantageous in designing a final product, as can alternative backbone structures like a polyamide backbone.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains.

A “ligand,” as used herein, is a molecule which binds to a particular receptor. The receptor may be a cell receptor or it may be a portion of another molecule, for example, a receptor for an allosteric modifier of an enzyme. Examples of ligands include, but are not limited to, enzyme cofactors, substrates and inhibitors, allosteric modifiers of enzymes, agonists and antagonists for cell membrane receptors, toxins and venoms, viral epitopes, haptens, hormones, lectins, and drugs such as opiates and steroids.

A “cell receptor,” as used herein, is a cellular molecule, which may be normally located either intracellularly or in association with the cell membrane, which has an affinity for a given ligand. Examples include, but are not limited to, hormone receptors, cellular transporters, cytokine receptors, and neurotransmitter receptors.

1.3 Immobilization of Probes

Oligonucleotide probes of the invention are affixed, immobilized, provided, and/or applied to the surface of the solid support using any available means to fix, immobilize, provide and/or apply oligonucleotides at a particular location on the solid support. The various species may be placed at specific sites using ink jet printing (U.S. Pat. No. 4,877,745), photolithography (See, U.S. Pat. Nos. 5,919,523, 5,837,832, 5,831,070, 5,770,722 and 5,593,839), silk printing, offset printing, stamping, mechanical application with micropipets using an x-y stage or other rastering technique, or any other method which provides for the desired degree of accuracy and spatial separation in placing the bound component.

Combinatorial array approaches, such as described by Southern et al. (U.S. Pat. Nos. 5,770,367, 5,700,637, and 5,436,327), Pirrung et al. (U.S. Pat. No. 5,143,854), Fodor et al. (U.S. Pat. Nos. 5,744,305 and 5,800,992), and Winkler et al. (U.S. Pat. No. 5,384,261), have been used with success in cases in which polymers of short sequences are required. In these “GeneChips,” oligonucleotide probes (20-25-mers) or peptide nucleic acids (PNAs) are produced either in situ during microarray fabrication, or offline using traditional methods and spotted on the microarrays. U.S. Pat. Nos. 5,445,934 and 5,744,305 to Fodor et al. describe the manufacture of substrates containing multiple sequences at density of 400 different probes per square centimeter or higher. These chip are synthesized using solid-phase chemistry and photolithographic technology. The combinatorial approaches generate significant biological and chemical diversity but are unable to construct microarrays of large macromolecules and can also be expensive and difficult to implement.

Ink jet dispenser devices are used to deposit small drops of liquid on a solid substrate. The fabrication of biological and chemical arrays by such technology has been shown by Brennan (U.S. Pat. No. 5,474,796), Tisone (U.S. Pat. No. 5,741,554), and Hayes et al. (U.S. Pat. No. 5,658,802). These non-contact technologies are unable to array large numbers of samples easily and to control the quality of the resultant microarrays.

A third category of arraying devices work by direct surface contact printing as described by Augenlicht (U.S. Pat. No. 4,981,783), Drmanac et al. (U.S. Pat. No. 5,525,464), Roach et al. (U.S. Pat. No. 5,770,151), Brown et al. (U.S. Pat. No. 5,807,522) and Shalon et al. (U.S. Pat. No. 6,110,426). In this format, the probes are long complementary DNAs (cDNAs) 500-5000 bases long, synthesized by traditional methods before immobilization. Deficiencies of such technologies as quill-based spotters include imprecise sample uptake and delivery as well as lack of durability.

Martinsky et al. (U.S. Pat. No. 6,101,946) describe the use of an electronic discharge machine (EDM) which can be attached to a motion control system for precise and automated movement in three dimensions. The oligonucleotide primers may also be applied to a solid support as described in Brown and Shalon, U.S. Pat. No. 5,807,522 (1998). Additionally, the primers may be applied to a solid support using a robotic system, such as one manufactured by Genetic MicroSystems (Woburn, Mass.), GeneMachines (San Carlos, Calif.) or Cartesian Technologies (Irvine, Calif.).

One may use a variety of approaches to bind an oligonucleotide to the solid substrate. By using chemically reactive solid substrates, one may provide for a chemically reactive group to be present on the nucleic acid, which will react with the chemically active solid substrate surface. One may form silicon esters for covalent bonding of the nucleic acid to the surface. Instead of silicon functionalities, one may use organic addition polymers, e.g. styrene, acrylates and methacrylates, vinyl ethers and esters, and the like, where functionalities are present which can react with a functionality present on the nucleic acid. Amino groups, activated halides, carboxyl groups, mercaptan groups, epoxides, and the like, may also be provided in accordance with conventional ways. The linkages may be amides, amidines, amines, esters, ethers, thioethers, dithioethers, and the like. Methods for forming these covalent linkages may be found in U.S. Pat. No. 5,565,324 and references cited therein.

One may prepare nucleic acids with ligands for binding and sequence tags by primer extension, where the primer may have the ligand and/or the sequence tag, or modified NTPs may be employed, where the modified dNTPs have the ligand and/or sequence tag. For RNA, one may use in vitro transcription, using a bacteriophage promoter, e.g. T7, T3 or SP6, and a sequence tag encoded by the DNA, and transcribe using T7, T3 or SP6 polymerase, respectively, in the presence of NTPs including a labeled NTP, e.g. biotin-16-UTP, where the resulting RNA will have the oligonucleotide sequence tag at a predetermined site and the binding ligand relatively randomly distributed in the chain.

In the present invention, probes may be synthesized in situ on the substrate or may be manufactured then immobilized on the substrate. This technique has been described for polynucleotides in U.S. Pat. No. 5,419,966, incorporated herein by reference. Alternatively, polymeric probes, such as polynucleotides, may be synthesized in a stepwise fashion from individual monomers or from smaller polynucleotides or other subunits. Preferably, probes are immobilized in discrete areas of the substrate. Alternatively, more than one probe can be immobilized in a particular area, with individual probe molecules of a particular type being distinguishable from other probe molecules by differential labeling, for example, with differently colored fluorescent tags. “Immobilize,” as used herein, means to attach a probe to the substrate by covalent or non-covalent means, with, sufficient affinity to withstand manufacturing, sample-binding, sample analysis steps, and, if necessary, re-use.

Methods and materials for derivatization of solid phase supports for the purpose of immobilizing polynucleotides and polypeptides are well-known in the art and are described in, for example, U.S. Pat. Nos. 5,744,305 and 5,919,523, which are hereby incorporated by reference in their entirety. For non-covalent attachment, the preferred method is by biotin-streptavidin attachment, but any method of non-covalent attachment that provides the necessary affinity is possible with the present invention.

In addition to oligonucleotides or any other organic entity, assemblages of molecules may also be used as in the case of organelles, e.g. nuclei, mitochondria, plastids, liposomes, etc., or cells, both prokaryotic and eukaryotic. The bound component may be directly bound to a solid substrate or indirectly bound, using one or more intermediates, which serve as bridges between the bound component and the solid substrate. In general, where a molecule is to be covalently bonded to the solid substrate surface, the surface may be activated using a variety of functionalities for reaction, depending on the nature of the bound component and the nature of the surface of the solid substrate.

For example, one may use a variety of approaches to bind the oligonucleotide to the solid substrate. By using chemically reactive solid substrates, one may provide for a chemically reactive group to be present on the nucleic acid, which will react with the chemically active solid substrate surface. One may form silicon esters for covalent bonding of the nucleic acid to the surface. Instead of silicon functionalities, one may use organic addition polymers, e.g. styrene, acrylates and methacrylates, vinyl ethers and esters, and the like, where functionalities are present which can react with a functionality present on the nucleic acid. Amino groups, activated halides, carboxyl groups, mercaptan groups, epoxides, and the like, may also be provided in accordance with conventional ways. The linkages may be amides, amidines, amines, esters, ethers, thioethers, dithioethers, and the like. Methods for forming these covalent linkages may be found in U.S. Pat. No. 5,565,324 and references cited therein.

One may prepare nucleic acids with ligands for binding and sequence tags by primer extension, where the primer may have the ligand and/or the sequence tag, or modified NTPs may be employed, where the modified dNTPs have the ligand and/or sequence tag. For RNA, one may use in vitro transcription, using a bacteriophage promoter, e.g. T7, T3 or SP6, and a sequence tag encoded by the DNA, and transcribe using T7, T3 or SP6 polymerase, respectively, in the presence of NTPs including a labeled NTP, e.g. biotin-16-UTP, where the resulting RNA will have the oligonucleotide sequence tag at a predetermined site and the binding ligand relatively randomly distributed in the chain. 1.4 Markers

The position of each probe on the substrate, as well as other information about the probe and/or the probe-sample complex, can be determined by using markers for probes or sets of probes. Such markers may be used with conventional two-dimensional arrays as well was with the present, one-dimensional configurations. “Markers,” as used herein, are any type of identifiable marking, arrangement, or other structure or pattern on, in, or associated with the substrate and/or probes which conveys information about a particular probe or set of probes. One type of marker can be optical. These can be space markers (i.e., breaks in the row of probes on the substrate, as described above) and/or bar codes, fluorescent markers, chemilluminescent markers, or any other marker capable of being detected with light. A further type of marker is magnetic markers. The present invention lends itself to such markers because the substrate may contain metallic elements which are magnetizable. They may be located on the same side of the substrate as the probes are, on the other side of the substrate from the probes, or may be sandwiched or otherwise incorporated into the interior of the substrate. One method for space marking and/or additional information is to coat the reverse side of the substrate from the probes with a layer of magnetic thin film. Then spatial or probe identification can be recorded during the fabrication process, by magnetic means. An important advantage of this approach is that additional information regarding the target can be written on to the substrate itself during the hybridization stage. And at the scanning stage, scanning parameters and other digital outputs may also be written on to the same tape for further reference. Other types of markers will be apparent to one of ordinary skill in the art. 1.5 Sets of Probes

Probes may be immobilized in sets on the substrate, each set sharing some common characteristic. For example, if probes are nucleotides, a group of nucleotides requiring common hybridization conditions may be immobilized along a certain length of substrate, and another group requiring a different set of hybridization conditions may be immobilized along another length of substrate. In this manner, each set of probes may be exposed to sample under a different set of conditions, optimizing sample binding.

Alternatively, a single probe carrier can carry different sets of probes for diagnosing different diseases. For example, one set of probes located along one stretch of a carrier might be used to diagnose HIV, which another set could be used to diagnose herpes, etc. As another example, a carrier or portion of a carrier could be devoted to the HER-2/neu gene. The HER-2 gene, also known as HER-2/neu and c-erbB2, plays a key role in the regulation of normal cell growth, but during the development of cancer, it becomes amplified. The amplified HER-2 gene results in the over-production of protein receptors found on the surface of tumor cells. These special proteins bind with other circulating growth factors to cause uncontrolled tumor growth. The probe carrier could contain probes for the HER-2/neu gene(s) and variations.

In another embodiment, the sets of probes may be redundant, allowing a single carrier to be used repeatedly for the same assay, with a new set of probes used for each successive assay. 1.6 Configuration of the Apparatus

Probes may be immobilized on the substrate in any configuration that allows one to distinguish and identify probes which have bound sample from those which have not.

The simplest way to do this is by placing probes in discrete areas, one probe per area. The areas may be spots, as shown in FIG. 1, or lines, as shown in FIG. 4, 404. The areas may be configured as a single row running along or parallel to the long axis of the substrate. The probes may be directly attached to the substrate, or, in an alternative embodiment, the probes may be attached to beads which are then attached to the substrate. Methods of attaching probes to beads of various materials are well-known in the art and are described in, for example, WO 99/60170, which is incorporated herein by reference in its entirety. Another alternative possible configuration is to have rows of spots, so that a plurality of probes is contained in a given row.

As illustrated in FIG. 1, DNA probes 100 are immobilized as spots at the center or as narrow stripes across the width of a long, thin and flexible thread substrate 100. Probe identification is achieved through space markers and/or bar codes 120 printed in the space 130 between groups of probes. Alternatively, these markers can be printed on the other side of the thread substrate. If necessary, the thread 100 can have a special cross-sectional shape. As shown in FIG. 2a, the cross-section of the fiber can be adjusted so that the fiber 200 has a notch or groove 202 in which probes 110 are immobilized. This design protects the probes of one layer from friction with the substrate of a succeeding layer, as shown in FIG. 11b, where the cross-sections of two successive layers are shown one on top of the other.

The long, thin, and flexible nature of the probe carrier lends itself to numerous novel means of containment, arrangement and use. The probe carrier may be packaged in a number of formats including but not limited to a pin, a rod, a coil and a spool. Hybridization methods are considerably enhanced by requiring less hybridization fluid and enhanced mixing. A flexible probe carrier packaged in a spool is especially advantageous in applications that require high volume, low to medium scale microarrays, such as those involved in disease diagnostics. In these applications, the required number of probes in the array may be small (in the range of several hundreds to several thousands) but a very large number of the same type of arrays may be available for consumption every day. With the flexible probe carrier format, tens of thousands of copies of identical sets of probes are arrayed repetitively along a continuous length of thread and sealed in a large coil or spool.

A pin or rod package is made by spirally winding a certain length of fabricated flexible probe carrier thread around a section of solid cylinder or tube. The thread sits tightly side-by-side on the outer surface of the supporting cylinder in the preferred embodiment and may be permanently attached to it by glue, cement or other means. The difference between the probe carrier pin and the probe carrier rod is the size. A probe carrier pin normally has a diameter less than 10 mm while a probe carrier rod is larger and can have a much larger diameter thus accommodating many more probes. For example, a 1.5 meter long, 50 μm diameter thread occupies only a short 5 mm section after being wound on a 5 mm diameter probe carrier pin, which may carry approximately 15,000 probes, presuming a 100 μm probe space along the thread. On the other hand, a probe carrier rod of 30 mm wide and 40 mm in diameter can accommodate as many as 700,000 probes along a 70 meter long, 50 μm diameter thread.

In a probe carrier coil, the fabricated flexible probe carrier thread is wound into a flat, disc shape coil. In one preferred embodiment of the invention, the probes on the thread are exposed on one side of the disc while the other side is permanently attached to a solid disc-shaped planar support by epoxy, cement or other suitable means. Optionally, probes are deposited in a notch on the surface of the probe carrier thread. The planar support can be pre-coated with a conductive layer to facilitate hybridization control. Assuming a 50 μm diameter thread, a probe carrier coil of 40 mm in diameter can accommodate up to 24 meters of probe carrier thread, carrying 240,000 probes.

The configuration of a probe carrier spool can be very similar to that of the probe carrier coil. However, unlike the probe carrier coil, the probe carrier thread is not permanently attached to a supporting surface, thus allowing the thread to unwind from the spool for hybridization, reading and other purposes. In addition, since each turn of the thread stacks on top of each other in the spool, the cross-section shape of the probe carrier thread can be designed to avoid friction between DNA probes and the probe carrier thread in an adjacent turn. Also shown in FIG. 17, a cassette can be constructed to protect the probe carrier spool and facilitate its winding and unwinding. In addition, multiple spools can be stacked up in one cassette.

2. Fabrication of the Probe Carrier

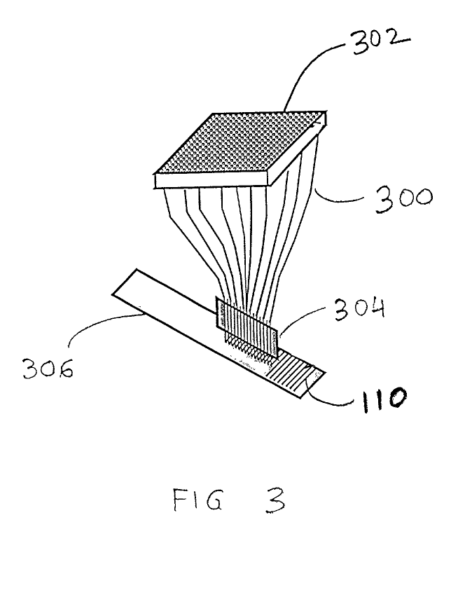

In certain embodiments of the probe deposition technology discussed herein, a fiber- or tape-like substrate is intrinsically suitable for continuous, high speed, mass production. FIGS. 3 through 7 show examples of fabrication systems designs. Although conventional spotting techniques may be used to produce discrete areas containing one probe each, one aspect of the present invention is the use of brushing or painting of probes on substrates. Such techniques, coupled with the essentially one-dimensional nature of the substrate, lend themselves to fabrication systems in which multiple copies of the same tape or fiber may be manufactured at once at high speed and with great precision.

2.1 Multi-stranded Brush

An exemplary embodiment of this apparatus and a method of manufacture is presented in FIG. 3. One method of manufacture, shown in FIG. 3, comprises transporting probes, which are either in a suitable liquid or are liquid themselves (for example, some lipids which are liquid at room temperature or can be liquefied at suitable temperatures), into tubing and depositing the probe from the tubing onto the substrate by moving the tubing relative to the substrate while at the same time driving the probe-liquid from the tube onto the substrate. It will be understood that such movements are relative and can be accomplished by moving the tubing assembly, moving the substrate, or both. During deposition, the tip of the capillary tube may contact the substrate surface. Alternatively, the tip may move a short distance above or underneath the substrate surface. The probe fluids are deposited onto the substrate using one of the non-contact deposition methods. These include attaching probes to magnetic beads suspended in the probe fluid and placing an electromagnet under the substrate. The magnet is activated during when the capillary and substrate intersect (i.e. the capillary passes in proximity to the substrate), which pulls the magnetic beads and their associated probe onto the substrate surface. Another non-contact deposition method is to coat a metal layer on both the end facet of the capillary tubing and either the substrate surface or the support under the substrate, then apply a high voltage between the capillary and the substrate or substrate support. The electric field will pull the electrically charged probes (such as oligonucleotides) onto the substrate surface. Using either of the above two methods, if the electric activation signal is a very short pulse, probe will be deposited on the substrate as a dot. If the signal is on for much or all of the time that the capillary and substrate intersect, the result will be a stripe of probe on the substrate. Conditions may be selected to ensure immobilization of the probe on the substrate. A plurality of tubes may be joined together to create a “brush” capable of depositing multiple probe stripes simultaneously. Further, a plurality of such brushes may be arranged to multiply the number of probes which may be deposited, either simultaneously or sequentially as different brushes move relative to the substrate and deposit probe stripes. In addition, if the substrate is a fiber, several fiber substrates may be positioned so that one stroke of a brush deposits probes stripes on all of the substrates. Alternatively, a wide tape substrate may be used to receive the probe stripes, then the tape may be cut lengthwise into a plurality of individual, thinner, tapes. It can be appreciated that such a method of manufacture greatly multiplies the number of probe carriers which may be produced simultaneously, increasing throughput and reducing cost. It can also be appreciated that standard mass production methods, such as the use of conveyor belts, can be readily adapted to automate and control this and other methods of manufacture presented herein.

In FIG. 3, a set of flexible capillaries 300 are glued into a hole under each well of a standard microtitre plate 302. A capillary 300 can also be inserted into the well from the top. The capillary 300 is then lined up into a linear array to form a “brush” 304. Each individual DNA probe is stored in a separate well in the plate and is driven into the capillary 300 linked to the well by pressure differentiation or by applying a voltage between the well and the tip of the brush 304. Because DNA molecules are negatively charged, a negative polarity should be applied to the well end. Multiple such capillary brushes can be constructed. After the capillaries are filled, the capillary array is moved to “brush” across a stationary probe carrier tape substrate 306 and deposit an array of DNA probes 110, then the tape substrate 306 will move forward to a new position to enable a second capillary “brush” 304 to deposit more probes 110 on subsequent positions. Alternatively, the same brush can be used to deposit more copies of the same probe array along the tape substrate 306. In additional, a large number of thread substrates can be laid in parallel under the brush so that each “brushing” action can produce multiple copies of the 1-dimensional probe array on different threads or tapes.

A further refinement of this technique, and of all the techniques presented, is to also deposit or establish markers for the probes 110 (see, for example, FIG. 1). Such markers may be spaces between or around probes, or they may be optical bar codes (FIG. 1, 120), or fluorescent markers, or magnetic markers encoded on a metallic element in the substrate, or any other means that would serve to identify a particular probe or group of probes. They may be established on the same side of the substrate that the probes are deposited on, on the opposite side, or both. It will be understood that a substrate may have only one surface (for example, a fiber with a circular cross-section), and that the term “side” in this context refers to the particular area of the surface on which probe is deposited. In the case of a tape, with a more defined top surface and bottom surface, “side” means one of these top or bottom surfaces.

A variety of means may be used to provide the force to drive probe from the reservoir, into the tubing, and onto the substrate. For example, a pressure differential may be established. Alternatively, if the probe is charged—as is the case with, for example, DNA—a voltage may be established between the reservoir and the substrate, such that the probe moves from the reservoir and tubing onto the substrate. The substrate may contain a metallic element such as a metal layer which forms an electrode.

2.2 Probe Printing Heads

FIG. 4 shows a second design of a probe carrier thread fabrication system, where each probe is stored in its own “printing head” 410 of a print system 408. A large number of such printing heads 410 are arranged into a one-dimensional array on a conveyer belt 400 moving at a constant speed Vₕ. The belt can wind across pulleys or capstans 412 into a spools 406 to conserve space. The spacing between the heads can be as large as several millimeters and be sufficient to accommodate a reservoir for each probe. A probe carrier tape substrate 402 is placed under the printing heads array, also moving at a constant, albeit slower speed Vₜ. When a printing head intersects the probe carrier tape substrate 402, it “prints” a spot or a stripe on the probe carrier tape substrate 402. Presuming the spacing between two adjacent printing heads on conveyer belt is Lₕ and the desired spacing between two adjacent probes on the probe carrier tape substrate 402 is Lₚ, the speeds of the printing head belt Vₚ and that of the probe carrier tape substrate 402, Vₜ, can be precisely controlled to satisfy Vₚ/Vₜ=Lₚ/Lₜ. In this way, a linear array of the DNA stripes 404 can be deposited on the probe carrier tape substrate 402 at high speed in a continuous fashion. The line may be diagonal across the substrate, since the substrate is moving, or the substrate and conveyor may intersect at an angle that results in a line of probe that is perpendicular to the long axis of the substrate. The substrate may instead be stopped while the print head prints, then advanced to the next printing position before the next probe is applied.

Each printing head in the system consists of a reservoir that holds a certain quantity of the probe sample and a means to transfer the probe onto probe carrier or tape thread substrate. Probes are dispersed in a liquid (or are themselves liquid) which provides the necessary conditions for transfer and immobilization of the probes on the substrate, the exact nature of which depends on the particular probe and the particular substrate.

FIG. 5 shows some possible designs for the printing heads. In FIG. 5a, a very thin, flexible fiber 500 is attached to a small opening 502 under a probe reservoir 504. The fiber 500 is hydrophilic so it draws the probe fluid onto its surface through surface tension or capillary effect. In addition, the fiber 500 has to be thin (<80 μm), flexible and yet not deform plastically. A solid or hollow silica fiber coated with metal or nylon is a good candidate. When intersected with the probe carrier tape substrate 402, it “draws” a stripe on the surface of the probe carrier tape substrate 402 using the probe as “ink”. In the case of metal coated fiber, a negative voltage can be applied to the fiber to push the DNA sample on to the probe carrier thread or tape.

FIGS. 5(b)-(h) show different designs based on the ink jet principle, where a pin hole is produced on the bottom of the probe reservoir. Pulse energy is introduced into the reservoir, which ejects droplets out of the pinhole on to the probe carrier thread underneath. In FIG. 5b, a piezo ring 506 is glued on the wall of the reservoir tube, which squeezes the tube under a voltage and ejects a droplet. In FIG. 5c, a piezo-film 506 is coated on a diaphragm 508 on top of the reservoir 504, which will have the same function as the piezo ring but could be less expensive when using a large number of reservoirs 504. In FIG. 5d, a current pulse through a resister wire 510 generates a bubble through localized heating, which in turn pushes out the droplet. In FIG. 5e, the ejecting energy is introduced through an external ultrasound transducer 512. In FIG. 5f, the reservoir tube is transparent and the heating is realized by focusing a laser 514 to a light absorption patch inside the tube. In FIG. 5g, the reservoir 516 is made of metal. A high voltage is applied between the reservoir and the probe carrier tape substrate 402 (or object underneath the probe carrier tape substrate 402) with the negative polarity on the reservoir. Since DNA carries negative charge, the electric field will eject the sample on to the probe carrier tape substrate 402 surface. In FIG. 5h, probe molecules are attached to magnetic beads suspended in fluid 520 within reservoir 518. A current pulse is applied to the electromagnet 519 underneath the substrate, which attracts the probe from the small opening under the reservoir onto the substrate surface 402. Note in design 5e to 5h, the actuators are external and do not move with the printing head. Since each reservoir (504 or 516 or 518) only intersects the probe carrier tape substrate 402 once at a fixed location, only one such actuator is needed in the system. Presuming a reservoir spacing of 2 mm, a 150,000 reservoirs array is 300 m long and can be accommodated in a spool less than 80 cm in diameter.

2.3 Spotters and Reservoirs

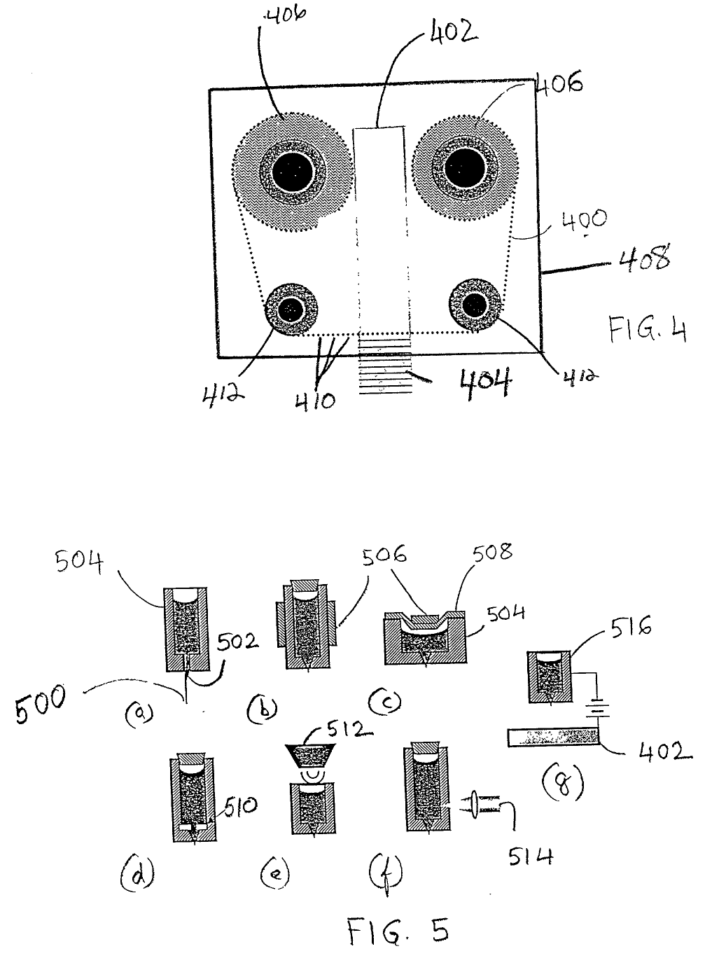

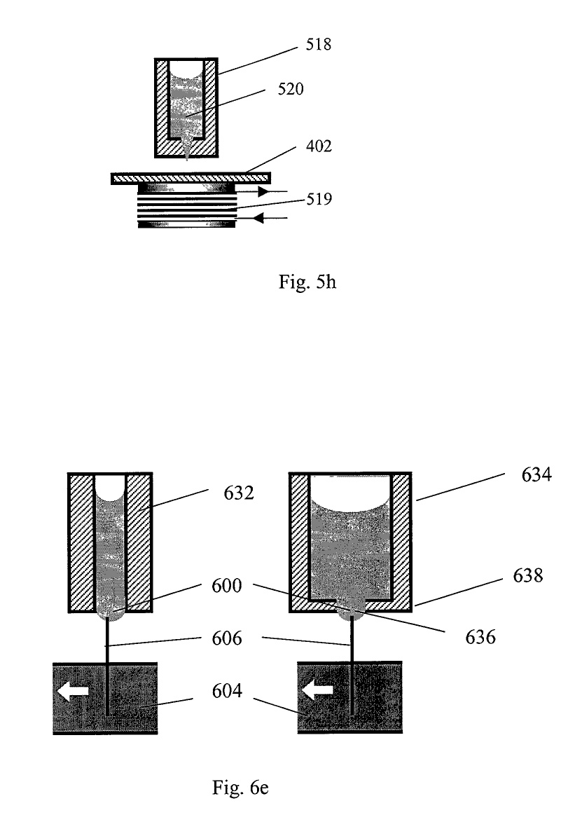

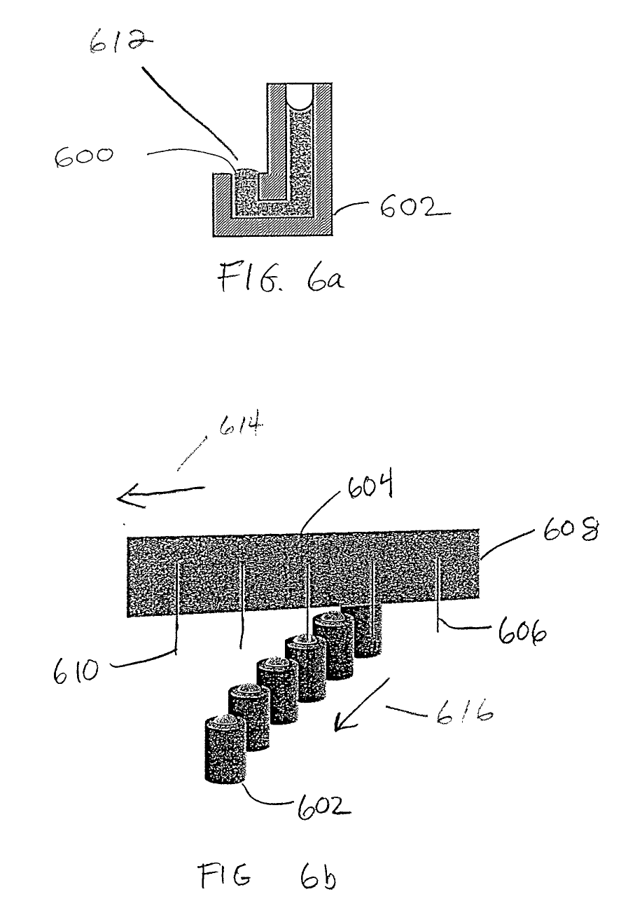

FIG. 6 illustrates another probe carrier fabrication system design, where the printing head configuration of the previous design is separated into a “spotter configuration” and a “reservoir configuration.” Each probe has its own reservoir, the structure of which is kept simple to reduce cost. FIG. 6a illustrates one of the possible reservoir designs, where the combination of liquid internal pressure and surface tension causes the liquid 600, which contains the probe 110, to bulge up a little at the opening 612. A large number of reservoirs 602 are assembled on a conveyer belt to form a linear array. As shown in FIG. 6b, the spotter configuration 604 can be fabricated by, for example, shaping a thin metal tape into a linear configuration of miniature teeth or gluing short silica fibers 606 to a flexible metal tape 608. Any material or combination of materials which produces a row of fibers suitable for transferring probe may be used. The tip of the spotter is suspended a short distance above the reservoir opening that allow the tip to contact the probe fluid. When the spotter is made of highly elastic materials, such as silica fiber, the spotter tip can actually slightly lower the opening so that the spotter can tip into the opening to collect the probe fluid. Both the spotter and reservoir configurations are driven to travel at e.g. a constant speed in different directions as indicated by arrows 614 and 616. When a spotter intersects opening 612 of a reservoir 602, a droplet of probe liquid 600 will be transferred from the reservoir to form droplet 610 on the spotter. The amount of liquid in the droplet can be controlled by the duration of the intersection and the shape and size of the spotter. The movement pattern of the spotter and reservoir configurations is designed in such a way (to be described later) that each consecutive spotter will intersect with corresponding consecutive reservoir so that each spotter now carries a different probe droplet. Then, as illustrated in FIG. 6c, the spotter configuration 604 moves on to intersect with a substrate 618 moving in a direction such that the spotter configuration and the substrate intersect. As with the reservoirs, the tip of each spotter 606 may physically contact the substrate or may be suspended a very short distance (several tens of micrometers) above the substrate, at a distance that allows droplet 610 to contact the substrate 618. The probe droplet will be transferred from the spotter configuration to the substrate to form a linear probe configuration 630 on the substrate. If the probe is charged, it is possible to charge a particular spotter by electronic means when it intersects a probe reservoir with a charge that will attract probe, then switch the charge on the spotter to the opposite charge when it later intersects the substrate, in order to repel the probe from the spotter and onto the substrate. This refinement allows the size of the droplet on the spotter to be controlled precisely with good consistency. Such a method to transfer probe material to the substrate is termed “spotting”, and is widely used manually in laboratories.

Alternatively, as shown in FIG. 6d, the spotter configuration 604 may move in a circle and the number of spotters in the configuration can be far fewer than the total number of probes. After the spotter leaves the substrate 618, it can be washed in a washing area 620, dried in a drying area 622 and reused by circling around to intersect 626 the probe reservoir configuration 624 again. Since the washing is carried out in parallel with spotting, it does not affect fabrication throughput. A spotter according to this invention is easy to clean

In an alternative configuration shown in FIG. 6e, the probe reservoir can be a straight tubing 632 or a well 634 with a small opening 636 at its bottom 638. The probe fluid 600 bulges downward at opening due to the combination of gravity and capillary force. The spotter 606 intersects the probe reservoir from underneath and collects some probe fluid 600 at its tip. Then the spotter moves on to intersect the probe carrier substrate 610 and paints a narrow stripe on the substrate in a configuration similar to FIG. 6c, except the spotter carrier 406 now passes under the substrate instead of above it. The substrate is positioned face down to be painted by spotters. The configuration of the entire fabrication system including probe collection, spotting, washing and drying is similar to that shown in FIG. 6d.

In the two fabrication systems described above, every component can move at a pre-defined, constant speed. This reduces the complexity of the motion control and precision requirement. In addition, a large number of probe carriers 100 can be manufactured continuously without manual intervention. As a result, the manufacturing throughput can be very high. Furthermore, if silica fiber or thin wire is adapted as the probe carrier substrate, many fibers can be attached in parallel to a wide carrier tape for the fabrication stage. So, multiple copies of the same probe carrier thread can be manufactured at the same time.

A wide tape substrate can be used on the fabrication station, with probe droplets being deposited in a line across the tape as illustrated in area 628 of FIG. 6d. The wide tape can be cut after the probe deposition to produce many copies of the same probe carrier threads 100, as illustrated in FIG. 1. In either case, the throughput can be further dramatically increased. These two system designs are therefore suitable for mass production at a dedicated central microarray fabrication facility. In one system of the invention, spacing between probes on the thread and that between reservoirs (and spotters) can be 100 μm and 5 mm, respectively. Assuming the thread substrate moves at a speed of 1 cm/s, the reservoir and spotter arrays travel at 50 cm/s, which is easily to achieve. Further, where the carrier tape 618 is separated into 20 thread substrates, the above two system designs should be capable of manufacturing a 150,000 probe array every 7.5 seconds.

2.4 Spotter Matrix

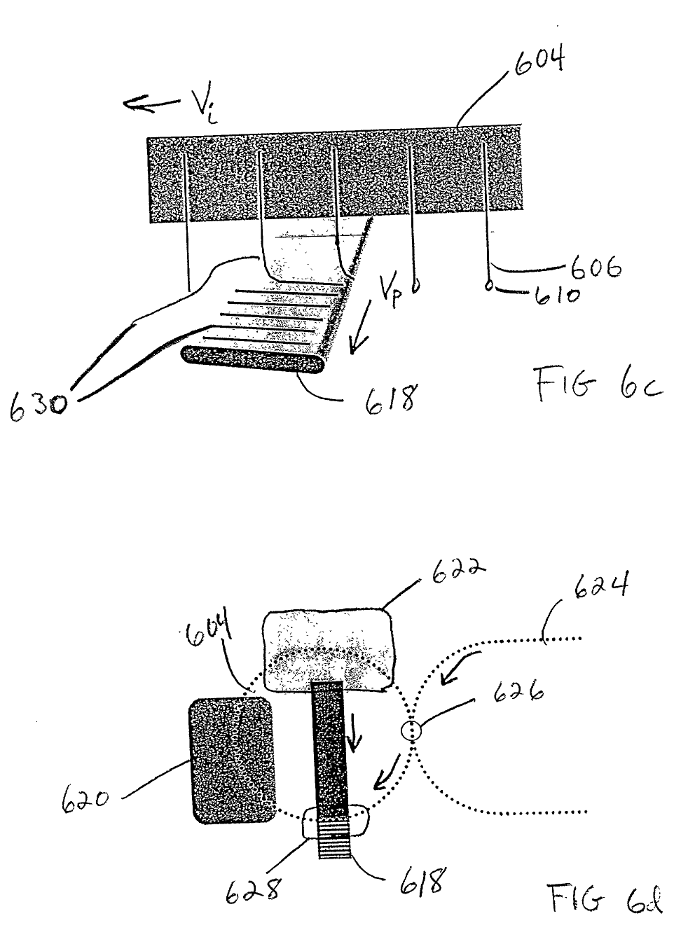

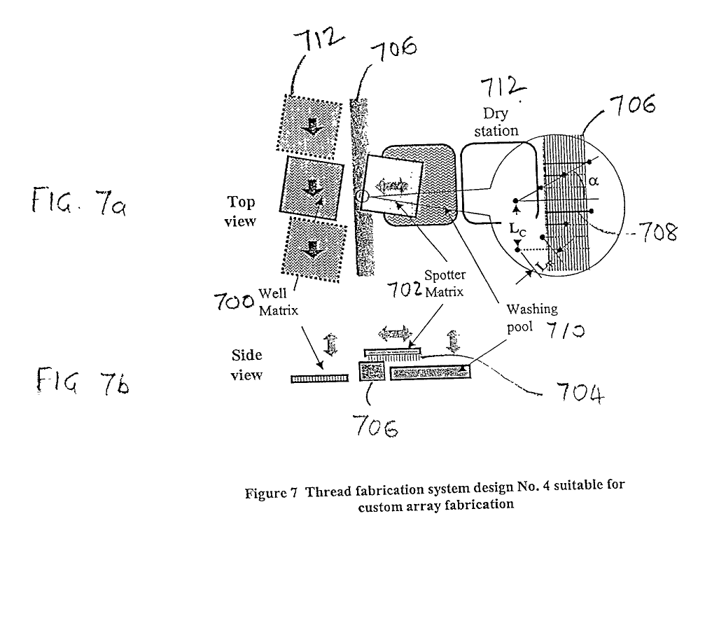

FIG. 7 shows a fourth fabrication system design, which has more flexibility and is particularly suitable for custom fabrication of smaller scale probe carriers. FIG. 7a is an overhead view and FIG. 7b is a frontal view of the system. Here, probes are stored in standard microtitre plates or similar matrix containers 700. A matching spotter matrix 702 has the same spacing as the well matrix in the plate. Different from conventional spotting pins, each spotter is a thin, flexible hydrophilic fiber 704, as used in the preceding system. The spotter matrix is first dipped into the well matrix, then moved to intersect the probe carrier tape substrate 706, which is temporarily held stationary. The direction that the spotter moves is perpendicular to the probe carrier tape substrate 706, but the direction of the matrix rows is tilted at a small angle of α. Each spotting fiber will produce a separate line 708 across the probe carrier tape substrate 706 (see enlarged view) for:

α=arc.sin[L_{c}/(C+1)L_{R]} (1)

where L_{c} and L_{R} are the fiber spacing in column and row of the spotter matrix, respectively and C is the number of columns in the spotter matrix.

The spotter matrix array is washed and cleaned in a cleaning area 710 and dried at a drying station 712. At the same time, the probe carrier tape substrate 706 advances to a fresh section and a new well matrix 712 is loaded, ready for the next “dipping and spotting”cycle. In this design, the spacing between probes on the probe carrier tape substrate 706 is given by L_{c}/(R+1), and can be about 250 μm. Such a density is useful for smaller scale custom arrays, as a probe carrier tape substrate 706 carrying 10,000 probes at a 259 urn probe spacing is 2.5 meters long, which can be wound into a spool less than 3 cm in diameter.

3. Packaging of Probe Carriers

The flexibility of the probe carrier thread platform enables the fabricated probe carrier thread to be packaged into a wide variety of different formats, which includes, but is not limited to, probe carrier pin, probe carrier rod, probe carrier coil and probe carrier spool. The superior strength, precision and flexibility of the probe carrier thread substrate are ideal for the precise probe positioning and transportation required in the probe carrier thread fabrication and reading process. Assuming both the probe spacing and thread thickness being 100 μm, the entire set of human genes (˜150,000) can be accommodated along a 15 m thread, which can be wound into a spiral coil 1.5 cm high and 3 cm in diameter or a spool of 0.1 mm thick and less than 4 cm in diameter. Thus probe carrier thread packaging is preferred for greater compaction of probes. Several modes of packaging a probe carrier thread and tape are described below.

3.1 Probe Carrier Pin and Probe Carrier Rod

As shown in FIG. 8, probe carrier pin 810 and probe carrier rod 820 are made by spirally winding a certain length of fabricated probe carrier thread 100 around a section of an elongated support member 804 such as a solid cylinder or tube. The tightly-wound thread 100 sits side-by-side 806 on a section 802 of the outer surface of the supporting cylinder 804 and may be permanently attached to it by glue, cement or other means. The cylinder 804 may be coated with conductive material before the winding process for hybridization control. The probes 110 are located on a side of the probe carrier thread 100 distal from the side of the probe carrier thread 100 which is contact with the support member 804.

As discussed previously, the difference between the probe carrier pin 810 and probe carrier rod 820 is the relative size and shape. A probe carrier pin 810 normally has a diameter less than 10 mm while a probe carrier rod 820 is larger and thus accommodates many more probes. For example, a 1.5 meter long, 50 μm diameter thread occupies only a short 5 mm section after being wound on a 5 mm diameter pin 810, which may carry approximately 15,000 probes, presuming a 100 μm probe space along the thread 100. On the other hand, a probe carrier rod 820 of 30 mm wide and 40 mm in diameter can accommodate as many as 700 k probes along a 70 meter long, 50 μm diameter thread 100. In one embodiment, the flexible probe carrier may be a tape substrate carrying probes immobilized in a 2-dimensional array. Fabrication of such arrays is illustrated in FIGS. 3 and 4 for instance. Such a flexible probe carrier tape can be wrapped around a pin 810 or a rod 820 instead of winding a probe carrier thread 100.

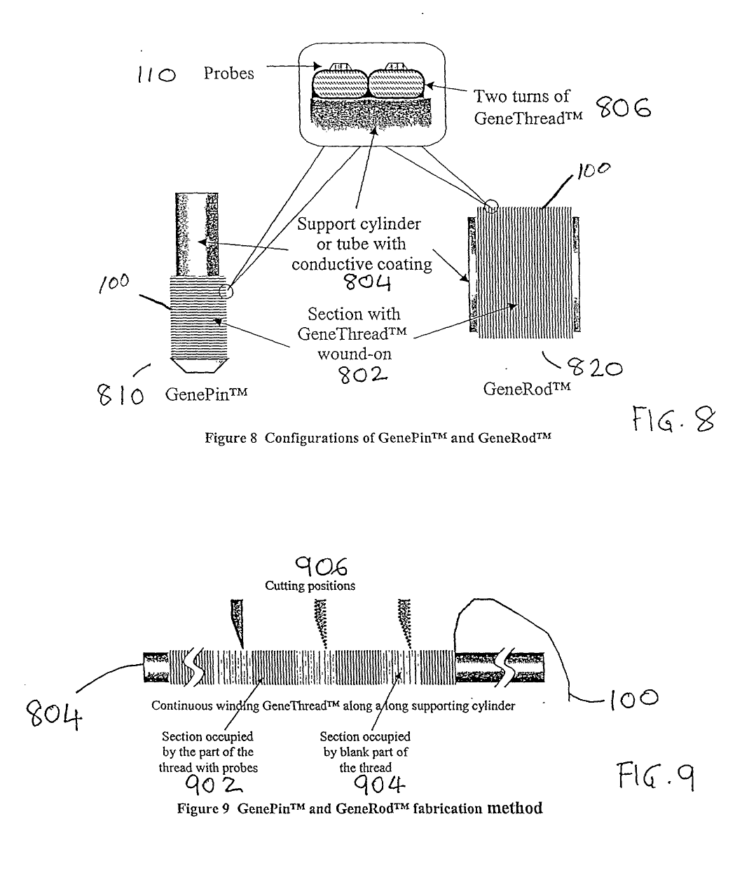

The probe carrier pin 810 and probe carrier rod 820 described above can be manufactured efficiently at a high throughput. As illustrated in FIG. 9, a certain length of “blank” space 904 is introduced between any two sets of probe arrays along the probe carrier thread or tape 100 during thread fabrication and prior to placing the thread or tape on the supporting cylinder. Then the probe carrier thread 100 is wound continuously along a long supporting cylinder 804. The cylinder 804 is pre-coated with epoxy or other adhesive at certain positions, where sections of the probe carrier thread 100 carrying probes 110 will be attached. After the epoxy is cured, the long cylinder 804 with thread 100 on can be cut at appropriate intervals to produce multiple probe carrier pins 810 or probe carrier rods 820 with probe carrier thread 100, with probes 110 attached, wound around the cylinder and at certain sections 902. Because the section 904 of the supporting cylinder 804, where the blank thread is attached to, is not pre-coated with epoxy, the blank thread will come loose and break off the cylinder after cutting, thus exposing a section 904 of the original supporting cylinder, which can be used to fit into adapters during the hybridization process.

3.2 Probe Carrier Coil

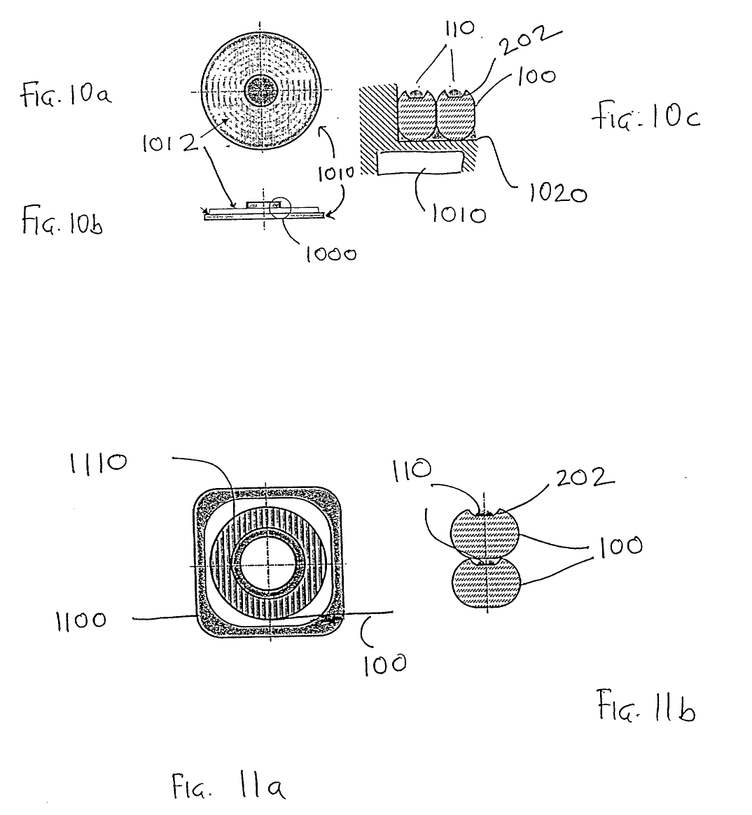

In a probe carrier coil shown in FIG. 10, the fabricated probe carrier thread 100 is wound into a flat, disc shape coil 1012. FIG. 10a shows a top view and FIG. 10b shows a side view of a probe carrier coil 1012 assembly. The probes 110 on the thread 100 are exposed on one side of the disc 1012 while the other side is permanently attached to a solid planar support disc 1010 by epoxy, cement or other suitable means. Note that in FIG. 10c, which illustrates an enlarged view of a cross-section 1000 of a probe carrier coil 1012, probes 110 are deposited in a notch 202 on the probe carrier thread 100 surface. This feature is optional in this packaging format. The planar support 1010 can be pre-coated with a conductive layer to facilitate hybridization control, which will be discussed in detail below. Presuming a 50 μm diameter thread, a probe carrier coil 1012 of 40 mm in diameter can accommodate up to 24 meters of thread, carrying 240,000 probes.

3.3 Probe Carrier Spool

The configuration of a probe carrier spool 1110 is very similar to that of the probe carrier coil. However, unlike probe carrier coil 1012, the probe carrier thread 100 is not permanently attached to a supporting surface 1010, thus allowing the thread to unwind from the spool for hybridization, reading and other purposes (although the end of the thread may be attached to the substrate). In addition, as shown in FIG. 11b, since each turn of the thread 100 stacks on top of each other in the spool 1110, the cross-sectional shape of the probe carrier thread 100 can be designed to avoid friction between DNA probes and the thread in adjacent turns. The cross-section of the substrate used to manufacture the probe carrier thread 100 can be selected such that the fiber 200 has a notch or groove 202 in which probes 110 are immobilized. This design protects the probes of one layer from friction with the substrate of a succeeding layer. Also, as shown in the FIG. 11a, a cassette 1100 can be constructed to protect the spool 1110 and facilitate its winding and unwinding. In addition, multiple spools 1110 can be stacked up in a single cassette 1100.

b 4. Hybridization

The use of an apparatus according to the present invention involves: 1) preparation of the sample; 2) formation of a probe-sample complex; and 3) analysis of the binding pattern in order to identify the individual probes to which sample has bound.

The preparation of the sample varies, depending on the sample type. Sample preparation protocols for analysis of polynucleotides, including labeling of samples with fluorescent tags in order to facilitate step 3), analyzing the binding pattern, are well-known in the art. See, for example, U.S. Pat. No. 5,800,992, which is hereby incorporated by reference in its entirety. In the case of polynucleotides, the sample is fragmented, using known techniques such as restriction endonuclease digestion, converted to single-stranded form, and the single-stranded fragments are labeled with an appropriate fluorescent tag.

Upon contact of sample with the apparatus, sample or sample fragments which have an affinity for particular probes bind with those probes. Present microarrays generally utilize hybridization of complementary strands of DNA as the binding method. DNA hybridization is highly dependent upon hybridization conditions, which have been extensively studied and described; see, for example, U.S. Pat. Nos. 6,054,270 and 5,700,637, which are hereby incorporated by reference in their entirety.

However, the present invention also encompasses any sort of sample-probe binding which will allow one to derive information from determining which probes have bound to sample or sample fragments. Examples include determining the identity of antigens or antibodies in a sample by using various antibodies or antigens, respectively, as probes, or identifying hormones in a sample by the receptors to which they bind, etc. The list of sample/probe pairs extends to any sets of pairs which bind with each other with a sufficient degree of affinity and specificity to be identified, and further examples of sample/probe pairs will be readily apparent to those of skill in the art.

Nucleic acid hybridization generally involves the detection of small numbers of target nucleic acids (DNA and RNA) among a large amount of non-target nucleic acids with a high degree of specificity. Stringent hybridization conditions are necessary to maintain the required degree of specificity and various combinations of agents and conditions such as salt, temperature, solvents, denaturants and detergents are used for the purpose. Nucleic acid hybridization has been conducted on a variety of solid support formats. (see, e.g., Beltz, G. A., et al., Methods in Enzymology, Vol. 100, part B, 19: 266-308, Academic Press, N.Y. (1985)).

Recent developments in DNA microarray technology make it possible to conduct a large-scale assay of a plurality of target molecules on a single solid phase support. Generally, a DNA chip including an oligonucleotide array is comprised of a number of individual oligonucleotides linked to a solid support in a regular pattern such that each oligonucleotide is positioned at a known location. After generation of the array, samples containing the target sequences are exposed to the array, hybridized to the complementing oligonucleotides bound to the array, and detected using a wide variety of methods, most commonly radioactive or fluorescent labels. U.S. Pat. No. 5,837,832 (Chee et al.) and related patent applications describe immobilizing an array of oligonucleotide probes for hybridization and detection of specific nucleic acid sequences in a sample.

This invention also provides some specially designed equipment for the hybridization of probe carrier thread based microarrays. In existing systems, hybridization is achieved by either natural diffusion or forced fluid circulation. The format is slow and the latter system is complicated to fabricate. In one embodiment of this invention, the hybridization chambers are designed to ensure that there is only a very thin layer of target fluid between the probe carrier thread or its packaged form and the inner wall of the hybridization chamber. In this way, only a very small volume of the target fluid is required for the hybridization, improving contact between probe molecules and target molecules. Hybridization acceleration is thus achieved by e.g. moving the probe carrier thread 100 or its packaged format through the target fluid.

In addition, the hybridization process can be further controlled by applying a voltage between the support of probe carrier thread and the inner wall of the hybridization chamber. During the process, hybridizations may also be accelerated by adding cations, volume exclusion or chaotropic agents. When an array consists of dozens to hundreds of addresses, it is important that the correct ligation product sequences have an opportunity to hybridize to the appropriate address. This may be achieved by the thermal motion of oligonucleotides at the high temperatures used, by mechanical movement of the fluid in contact with the array surface, or by moving the oligonucleotides across the array by electric fields. After hybridization, the array is washed sequentially with a low stringency wash buffer and then a high stringency wash buffer.

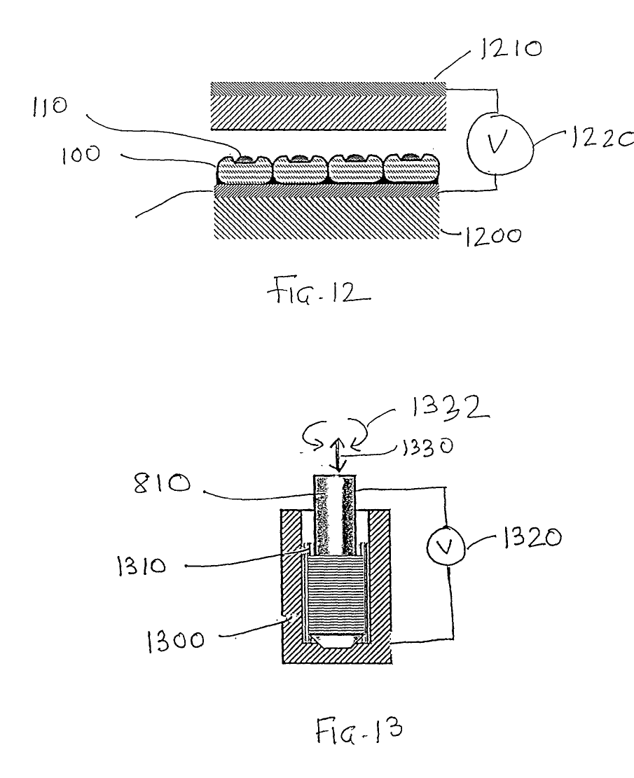

As shown in FIG. 12, when the probe carrier thread 100 has positive polarity, the DNA molecules in the target fluid are attracted towards the probe carrier thread 100, creating a temporary localized concentration near the thread surface to enhance hybridization. If the polarity is reversed, the electric field will repulse mismatched nucleic acid molecules away from the probes 110 while hybridized probes retain their target molecules, thus increasing the specificity of hybridization. Therefore, an AC oscillation voltage 1220 can be applied between the probe carrier thread 100 or its support 1200 and the wall of the hybridization chamber 1210 to improve the efficiency of the process. The support 1200 and the wall of the hybridization chamber 1210 have conductive coating 1212 in order to facilitate the process. As described below, all probe carrier thread formats may also rotate during hybridization, increasing agitation and mixing and thus improving contact between probes and target molecules. A brush slip ring can be used to conduct voltage on to the moving electrode. The design of such electric slip ring is well known in the art.

As shown in FIG. 13, a probe carrier pin 810 can be hybridized by directly plugging into a well 1300 containing target fluid 1310. The diameter of the well 1300 is only slightly larger than the outer diameter of the probe carrier pin 810. As there is only a very thin layer of target fluid 1310 between the probe carrier pin 810 and the inner wall of the fluid well 1300, the required volume for the target fluid is minimal. For example, presuming the well is 8 mm in diameter and the probe carrier pin is only 50 μm smaller in diameter and the wound section is 5 mm high, 3 μl target fluid would be sufficient to cover the entire effective section of the probe carrier pin. The probe carrier pin can undergo an up-and-down translational 1330 or back-and-forth 1332 rotational motion, or the combination of the two, in order to increase the hybridization speed. Because of the spiral winding pattern on probe carrier pin 810, probe carrier rod 820 and probe carrier coil 1012, rotational motion drives the target fluid along not only the circular direction but also the axial or radial directions of the package. It efficiently moves the molecules in the target over the entire surface area covered by probe carrier thread 100.

Multiple probe carrier pins 810 can be plugged into an adapter frame 1400 to form a matrix that is compatible with the spatial pitch and pattern of a standard microtiter plate 1420. In this way, multiple hybridization processes can be carried out in parallel directly in a standard microtiter plate 1420 by dipping each probe carrier pin 810 into a corresponding well 1410 of the standard microtiter plate 1420, as shown in

FIG. 14, and optionally translating the adapter plate up and down or rotating the individual probe carrier pins.

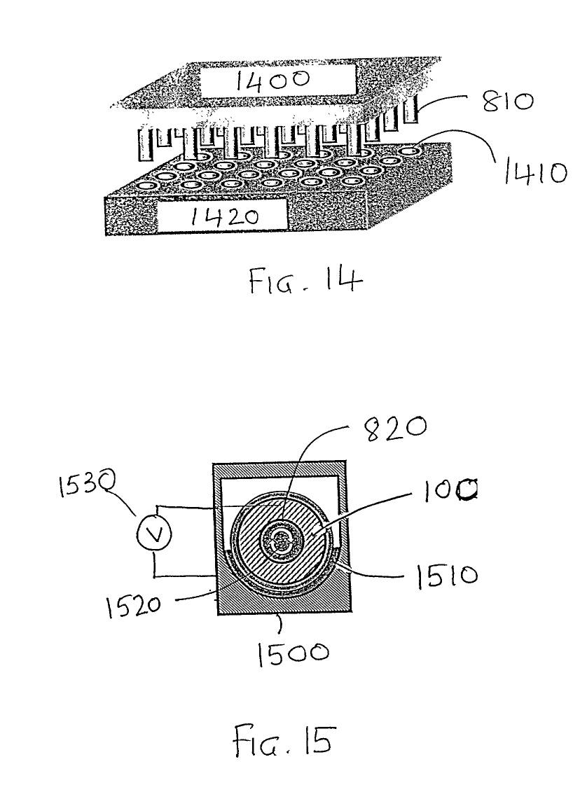

A probe carrier rod 820 can be hybridized in a similar, albeit larger hybridization chamber as that of probe carrier pin. Alternately, a chamber design 1500 shown in FIG. 15 can be used. A probe carrier rod 820 is rotated 1520 to move the target fluid 1510 over the probes. Because of the spiral winding pattern of the probe carrier thread 100 on the probe carrier rod 820, target fluid 1510 can be moved not only along the circular but also axial direction of the rod 820, thus covering all probe positions on the probe carrier rod 820. An AC oscillation voltage 1530 can be applied between the probe carrier thread 100 and the wall of the hybridization chamber 1500 to improve the efficiency of the process.

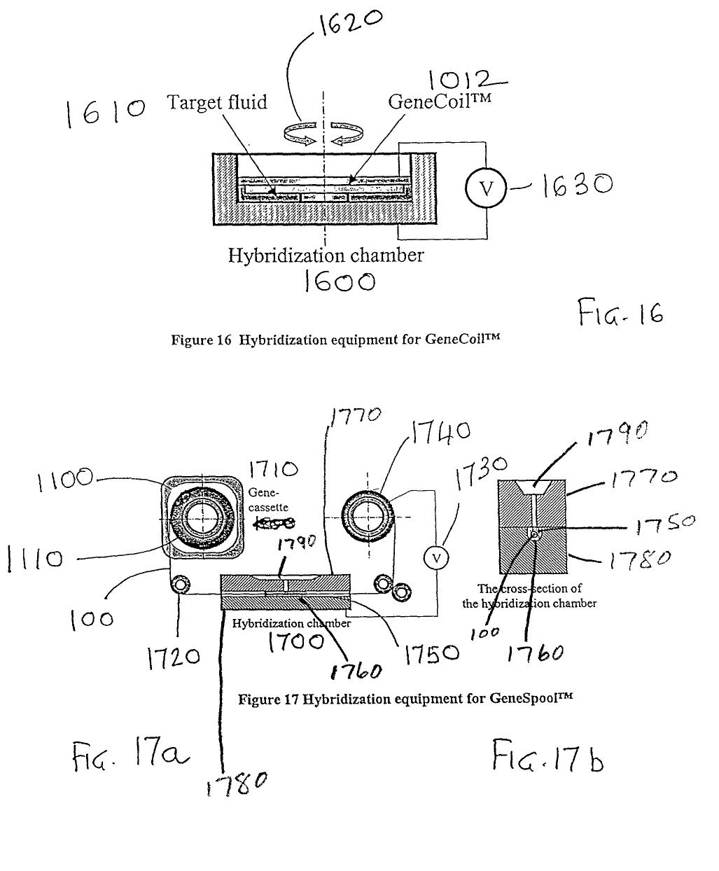

A hybridization chamber design 1600 for probe carrier coil 1012 is shown in FIG. 16. Again, a back and forth rotational motion 1620 is introduced to the coil through a mechanical drive or a magnetic drive and AC oscillation voltage alteration 1630 is applied between the coil support and the chamber to enhance the hybridization efficiency.

FIG. 17a shows a chamber design 1700 for the hybridization of probe carrier thread 100 that is unwound from a probe carrier spool 1110, in which a mostly water-tight capillary 1760 is formed by closing a lid 1770 on a narrow slot produced on a substrate 1780. The cross-sectional size of the slot is slightly larger than the probe carrier thread as shown in FIG. 17b. Target fluid 1750 is introduced into the middle section of slot before closing the lid or it is introduced through a small opening 1790 in the lid after the lid is closed onto the slot. The probe carrier thread 100 is moved back-Page-and-forth through the chamber to enhance the efficiency of the hybridization. As the thread is hydrophobic, the target fluid is retained inside the slot by the capillary force. Again, the hybridization efficiency can be further improved by applying an alternating voltage 1730 between a metal layer on the probe carrier thread 100 and the inner wall of the capillary 1760 of chamber 1700.

5. Reader

All probe carrier thread packaging formats described above can be read using a scanning microscope with laser or broadband excitation. Scanning can be carried out by scanning electron microscopy, confocal rnicroscopy, a charge-coupled device, scanning tunneling electron microscopy, infrared microscopy, atomic force microscopy, electrical conductance, and fluorescent or phosphor imaging. However, special scanning motions may preferably be provided in the readout instrument for various probe carrier thread formats.

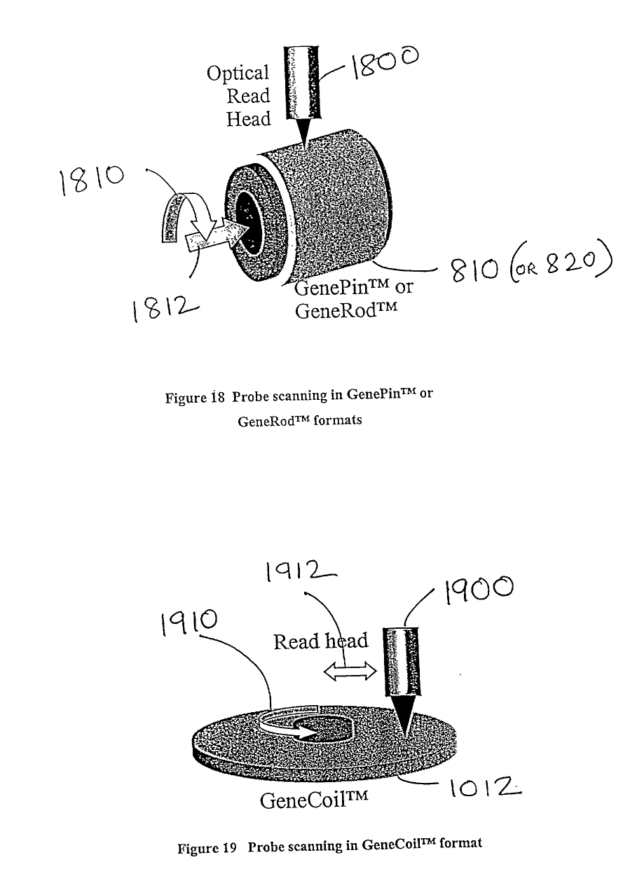

As illustrated schematically in FIG. 18, both probe carrier pin 810 and probe carrier rod 820 can be plugged into an adapter in the readout instrument designed to hold the ends of the pin or rod and rotate 1810 and/or translate 1812 them along the longitudinal axis at a predetermined ratio of speeds. This motion brings all probes distributed along the probe carrier thread 100 under the optical excitation and readout lens 1800. Alternatively, the probe carrier pin 810 or probe carrier rod 820 may rotate 1810 while the optical head 1800 translates along the axis of the pin or rod to scan the length of the probe carrier thread 100 mounted on a probe carrier pin 810 or a probe carrier rod 820.

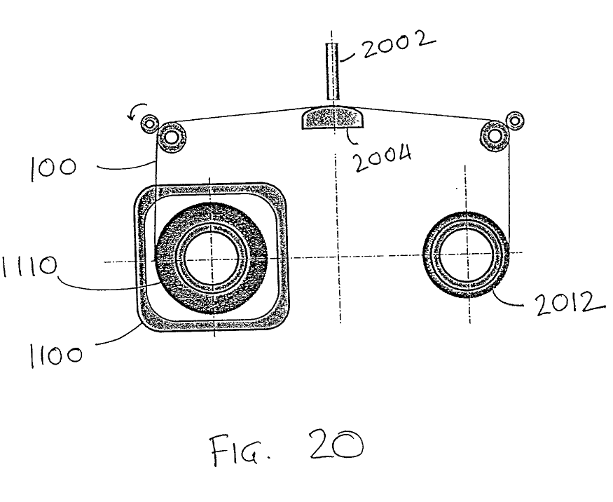

Similarly, as shown in FIG. 19, probe carrier coil 1012 can be scanned by introducing a rotation 1910 of the coil 1012 and a relative translation 1912 motion between the coil 1012 and the optical read head 1900 along a radial direction of the coil.

In probe carrier spool scanner illustrated in FIG. 20, a probe carrier spool 1110 contained in a cassette 1100 unwinds a stretch of unwound probe carrier thread 100 which is passed under an optical read head 2002 and a marker reader 2004. The unwound probe carrier thread 100 carries the entire set of probes moving under the optical read head 2002, which can remain stationary. The unwound probe carrier thread 100 can be collected in a second spool 2012.

6. Methods of Using Probe Carriers

The apparatus lends itself to use in a number of fields. An apparatus which uses polynucleotides as probes may be used for analysis of known point mutations, genomic fingerprinting, linkage analysis, characterization of mRNAs and mRNA populations, sequence determination, disease diagnosis, and polymorphism analysis. An apparatus which uses antibodies as probes would be especially useful in diagnostics. Other uses involving other probes will be apparent to those of skill in the art.

The use of the apparatus involves: 1) Preparation of the sample, if necessary; 2) Formation of probe-sample complex; 3) Analyzing the binding pattern in order to identify the individual probes to which sample has bound.

6.1. Preparation of the Sample

The preparation of the sample will vary, depending on the sample type. Sample preparation protocols for analysis of polynucleotides, including labeling of samples with fluorescent tags in order to facilitate step 3), analyzing the binding pattern, are well-known in the art. See, for example, U.S. Pat. No. 5,800,992, which is hereby incorporated by reference in its entirety. In the case of polynucleotides, the sample is fragmented, using known techniques such as restriction endonuclease digestion, converted to single-stranded form, and the single-stranded fragments are labeled with an appropriate fluorescent tag.

6.2 Formation of the Probe-sample Complex

Upon contact of sample with the apparatus, sample or sample fragments which have an affinity for particular probes bind with those probes. Present microarrays generally utilize hybridization of complementary strands of DNA as the binding method. DNA hybridization is highly dependent upon hybridization conditions, which have been extensively studied and described; see, for example, U.S. Pat. Nos. 6,054,270 and 5,700,637, which are hereby incorporated by reference in their entirety.

However, the present invention also encompasses any sort of sample-probe binding which will allow one to derive information from determining which probes have bound to sample or sample fragments. As an example only, the sample may be composed of a number of molecules, some of which are enzymes. The probes of the apparatus to be used to analyze this sample could be substrates for various enzymes (here the word “substrate” is used in the sense of a reactant upon which an enzyme works as a catalyst), and the identity of the enzymes in the samples may be obtained by determining which substrate-probes have bound enzymes after contact. Other examples include determining the identity of antibodies in a sample by using various antigens as probes, or identifying hormones in a sample by the receptors to which they bind, etc. The list of sample/probe pairs extends to any sets of pairs which bind with each other with a sufficient degree of affinity and specificity to be identified, and further examples of sample/probe pairs will be readily apparent to those of skill in the art.

One aspect of the present invention can greatly enhance binding of charged sample. This is the ability to supply a voltage across the substrate, where the substrate contains a metallic element or is otherwise electrically conductive. For example, if DNA is the sample to be analyzed, an oscillating voltage across the substrate will alternately attract the negatively charged DNA to the probe carrier, then repel it. The attraction will facilitate the binding of complementary strands, while the repulsion cycle will expedite the release of non-specifically bound or incompletely hybridized sample. The same principle holds true for any type of charged sample, and increases the efficiency and fidelity of sample binding.

6.3 Analysis of the Binding Pattern

There are generally two steps in the analysis of the binding pattern: locating the probes which have bound sample, and identifying what those probes are. It is possible that for a particular sample/probe pair the two steps may reduce to one, if binding of a particular sample to its corresponding probe produces a change which is unique to that sample/probe pair.

Distinguishing probe-sample pairs from probes which have not bound sample may be done in any manner that allows localization. Many such techniques are well-established in the art. Detecting labeled sample polynucleotides, for example, can be conducted by standard methods used to detect the type of label used. Thus, for example fluorescent labels or radiolabels can be detected directly. Other labeling techniques may require that a label such as biotin or digoxigenin that is incorporated into the sample during preparation of the sample and detected by an antibody or other binding molecule (e.g. streptavidin) that is either labeled or which can bind a labeled molecule itself, for example, a labeled molecule can be e.g. an anti-streptavidin antibody or anti-digoxigenin antibody conjugated to either a fluorescent molecule (e.g. fluorescein isothiocyanate, Texas red and rhodamine), or conjugated to an enzymatically active molecule. Whatever the label on the newly synthesized molecules, and whether the label is directly in the sample or conjugated to a molecule that binds the sample (or binds a molecule that binds the sample), the labels (e.g. fluorescent, enzymatic, chemilluminescent, or colorimetric) can be detected by a laser scanner or a CCD camera, or X-ray film, depending on the label, or other appropriate means for detecting a particular label. For example, in most uses of microarrays of polynucleotides for gene analysis, sample polynucleotides are fragmented, then each sample fragment is tagged with a fluorescent label. Following contact with the probe polynucleotide array, the sample fragments which have hybridized with complementary probe polynucleotides may be located by the fluorescent tag on the sample. Probes which have not bound sample fragments have no such fluorescent label. Similar fluorescent tagging may be done for other types of molecules, such as antibodies, enzymes, etc. Other types of tags, such as radioactive labels, chemilluminescent labels, phosphorescent labels, magnetic labels, etc., will be readily apparent to one of skill in the art.

For detection of probes that have bound sample, light detectable means are preferred, although other methods of detection may be employed, such as radioactivity, atomic spectrum, and the like. For light detectable means, one may use fluorescence, phosphorescence, absorption, chemilluminescence, or the like. The most convenient will be fluorescence, which may take many forms. One may use individual fluorescers or pairs of fluorescers, particularly where one wishes to have a plurality of emission wavelengths with large Stokes shifts (at least 20 nm). Illustrative fluorescers include fluorescein, rhodamine, Texas red, cyanine dyes, phycoerythrins, thiazole orange and blue, etc. When using pairs of dyes, one may have one dye on one molecule and the other dye on another molecule which binds to the first molecule. The important factor is that the two dyes when the two components are bound are close enough for efficient energy transfer.

The present invention provides opportunities for greatly streamlining and expanding the second step in the analysis, that is, the step of identifying the specific probes to which samples or sample fragments have bound. In conventional probe microarrays, such as polynucleotide arrays, the identity of a probe is established by determining its x-y position in the array; the x-y position of every probe is known. Determining the position of the probe by known techniques requires complex and expensive imaging equipment. Because the probes in the present invention are arranged in a one-dimensional row, positional analysis is much easier and requires much less complex equipment, because only one dimension need be tracked (as is the case for a spooled thread), rather than two.

The use of markers associated with probes or groups of probes provides a means for keeping track of probes in any of the embodiments of the invention. This has been discussed previously. Markers may be simple or complex, may be on the same side of the substrate as the probes or a different side, may be of more than one type, and may contain more information than just the identity of the probe or probes.

Probe carriers of the present invention can be used to construct very large probe arrays packaged in minimal volume which are subsequently hybridized with a target nucleic acid. Analysis of the hybridization pattern of the chip provides an immediate fingerprint identification of the target nucleotide sequence. Patterns can be manually or computer analyzed, but it is clear that positional sequencing by hybridization lends itself to computer analysis and automation. Algorithms and software have been developed for sequence reconstruction which are applicable to the methods described herein (R. Drmanac et al., J. Biomol. Struc. & Dyn. 5:1085-1102, 1991; P. A. Pevzner, J. Biomol. Struc. & Dyn. 7:63-73, 1989, both of which are herein specifically incorporated by reference).

Flexible probe carriers containing immobilized nucleic acid sequences prepared in accordance with the invention can be used for large scale hybridization assays in numerous genetic applications, including analysis of known point mutations, genomic fingerprinting, linkage analysis, characterization of mRNAs and mRNA populations, sequence determination, disease diagnosis, and polymorphism analysis. An apparatus which uses antibodies as probes would be especially useful in diagnostics. Other uses involving other probes will be apparent to those of skill in the art.

For gene mapping, a gene or a cloned DNA fragment is hybridized to an ordered array of DNA fragments, and the identity of the DNA elements applied to the array is unambiguously established by the pixel or pattern of pixels of the array that are detected. One application of such arrays for creating a genetic map is described by Nelson, et al., Nature Genetics 4:11-18 (1993). In constructing physical maps of the genome, arrays of immobilized cloned DNA fragments are hybridized with other cloned DNA fragments to establish whether the cloned fragments in the probe mixture overlap and are therefore contiguous to the immobilized clones on the array. For example, Lehrach et al., “Hybridization Fingerprinting in Genome Mapping and Sequencing,” in Genome Analysis, vol. 1: Genetic and Physical Mapping. (K. E. Davies & S. M. Tilghman, Eds.) Cold Spring Harbor Laboratory Press, pp. 39-81 (1990), describe such a process.

Flexible probe carriers of immobilized DNA fragments may also be used for genetic diagnostics. To illustrate, a probe carrier containing multiple forms of a mutated gene or genes can be probed with a labeled mixture of a patient's DNA which will preferentially interact with only one of the immobilized versions of the gene. The detection of this interaction can lead to a medical diagnosis. Also, detection of expression levels of certain genes are diagnostic of certain medical conditions. For example, amplification of the HER-2/neu (c-erbB-2) gene resulting in overexpression of the p185HER-2 growth factor receptor occurs in approximately 25% of early stage breast cancers. HER-2/neu has been established as an important independent prognostic factor in early stage breast cancer in large cohorts of patients and in cohorts with very long (30 year) follow-up duration. New data has emerged to suggest that HER-2/neu may be useful not only as a prognostic factor but also as a predictive marker for projecting response to chemotherapeutics, antiestrogens, and therapeutic anti-HER2/neu monoclonal antibodies. HER-2/neu codes for a 185 kD transmembrane oncoprotein which is amplified and/or over-expressed in some breast cancer patients, a feature generally associated with a poorer prognosis than that for women with unamplified HER-2/neu. While this locus has been studied for a number of years, technical problems associated with the most commonly used methodologies (Southern blotting and immunohistochemical staining) have led to some inconsistencies in the data. Pegram M. D. et al., HER-2/neu as a predictive marker of response to breast cancer therapy. Breast Cancer Research and Treatment 52(1-3): 65-77, 1998. The rapid processing of multiple samples enabled by the present invention, allow for rapidly testing multiple controls to avoid inconsistencies.

7. Utilities of Probe Carriers

Flexible probe carriers of immobilized DNA fragments can also be used in DNA probe diagnostics. For example, the identity of a pathogenic microorganism can be established unambiguously by hybridizing a sample of the unknown pathogen's DNA to a probe carrier containing many types of known pathogenic DNA. A similar technique can also be used for unambiguous genotyping of any organism. Other molecules of genetic interest, such as cDNAs and RNAs can be immobilized on the probe carrier or alternately used as the labeled probe mixture that is applied to the probe carrier.

In one application, a probe carrier of cDNA clones representing genes is hybridized with total cDNA from an organism to monitor gene expression for research or diagnostic purposes. Labeling total cDNA from a normal cell with one color fluorophore and total cDNA from a diseased cell with another color fluorophore and simultaneously hybridizing the two cDNA samples to the same array of cDNA clones allows for differential gene expression to be measured as the ratio of the two fluorophore intensities. This two-color experiment can be used to monitor gene expression in different tissue types, disease states, response to drugs, or response to environmental factors.

By way of example and without implying a limitation of scope, such a procedure could be used to simultaneously screen many patients against all known mutations in a disease gene. This invention could be used in the form of, for example, 96 identical probe carrier pins in a matrix where each probe carrier pin could contain, for example, 1500 DNA fragments representing all known mutations of a given gene. The region of interest from each of the DNA samples from 96 patients could be amplified, labeled, and hybridized to the 96 individual arrays with each assay performed in 10 microliters of hybridization solution. The adapter matrix containing all 96 identical probe carrier pins assayed with the 96 patient samples is incubated, rinsed, detected and analyzed as a single sheet of material using standard radioactive, fluorescent, or colorimetric detection means (Maniatis, et al., 1989). Previously, such a procedure would involve the handling, processing and tracking of 96 separate membranes in 96 separate sealed chambers. By processing all 96 patient samples in a single step with minimal hybridization liquid, significant time and cost savings are possible.

The assay format can be reversed where the patient or organism's DNA is immobilized as the probe elements and each probe carrier is hybridized with a different mutated allele or genetic marker. A probe carrier matrix can also be used for parallel non-DNA ELISA assays. Furthermore, the invention allows for the use of all standard detection methods.

One aspect of this invention involves the detection of nucleic acid sequence differences using coupled ligase detection reaction (LDR) and polymerase chain reaction (PCR) as disclosed in U.S. Pat. No. 6,027,889 entitled “Detection of nucleic acid sequence differences using coupled ligase detection and polymerase chain reactions” to Baranyi, et al. which is incorporated herein by reference in its entirety.

In addition to the genetic applications listed above, arrays of whole cells, peptides, enzymes, antibodies, antigens, receptors, ligands, phospholipids, polymers, drug cogener preparations or chemical substances can be fabricated by the means described in this invention for large scale screening assays in medical diagnostics, drug discovery, molecular biology, immunology and toxicology.

All publications and patent applications mentioned in this specification are incorporated herein by reference to the same extent as if each individual publication or patent application were specifically and individually indicated to be incorporated by reference.

The foregoing description of preferred embodiments of the invention has been presented by way of illustration and example for purposes of clarity and understanding. It is not intended to be exhaustive or to limit the invention to the precise forms disclosed. It will be readily apparent to those of ordinary skill in the art in light of the teachings of this invention that many changes and modifications may be made thereto without departing from the spirit of the invention. It is intended that the scope of the invention be defined by the appended claims and their equivalents.

What is claimed is:

1. An apparatus for allowing specific identification of samples with probes, comprising
a flexible elongated substrate having a first substrate surface, a length, and a width; and
a plurality of non-identical probes immobilized on discrete areas of a probe-containing portion of the substrate surface, each of said discrete areas containing one probe.

2. The apparatus of claim 1 wherein each discrete area containing one probe has a length not exceeding 500 micrometers.

3. The apparatus of claim 1 wherein each discrete area containing one probe has a length not exceeding 100 micrometers.

4. The apparatus of claim 1 wherein each discrete area containing one probe has a length not exceeding 50 micrometers.

5. The apparatus of claim 1 wherein each discrete area containing one probe has a length not exceeding 20 micrometers.

6. The apparatus of claim 1 wherein the probes are selected from the group consisting of polynucleotides, polypeptides, polysaccharides, and lipids.

7. The apparatus of claim 1 wherein the substrate is made of materials selected from the group consisting of silica, glass optical fibers, metals, magnetizable materials, plastics, polymers, polyimide, and polytetrafluoroethylene.

8. The apparatus of claim 1 further comprising a first marker which conveys information about a first set of said probes and a second marker which conveys information about a second set of said probes.

9. The apparatus of claim 1 wherein the ratio of the length to the width of the substrate exceeds 5:1.

10. The apparatus of claim 1 wherein the ratio of the length to the width of the substrate exceeds 100:1.

11. The apparatus of claim 1 wherein the ratio of the length to the width of the substrate exceeds 10,000:1.

12. The apparatus of claim 1 wherein the ratio of the length to the width of the substrate exceeds 100,000:1.

13. An apparatus for allowing specific identification of samples with probes, comprising
a flexible elongated substrate having a substrate surface, a length, and a width;
a first layer on the surface of the substrate; and
a plurality of non-identical probes immobilized on a probe-containing portion of the surface of said layer, said probe-containing portion having a length and a width such that the ratio of the length of the probe-containing portion to the width of the probe-containing portion exceeds 5:1.

14. The apparatus of claim 13 further comprising a second layer between said first layer and said substrate.

15. The apparatus of claim 14 wherein said first layer comprises silica and said second layer comprises a metallic material.

16. A linear one-dimensional arrangement of probes, comprising
a flexible substrate having at least a first surface; and
a plurality of probes immobilized on the first surface of the substrate and arranged in a single-file row at a linear density exceeding 50 probes/linear cm.

17. The arrangement of claim 16, wherein the linear density of probes arranged in a single-file row on the substrate exceeds 100 probes/linear cm.

18. The arrangement of claim 16, wherein the linear density of probes arranged in a single-file row on the substrate exceeds 200 probes/linear cm.

19. The arrangement of claim 16, wherein the linear density of probes arranged in a single-file row on the substrate exceeds 500 probes/linear cm.

20. A probe-carrying tape apparatus that is configured to bind samples to form sample-probe complexes, said tape comprising
a flexible tap e substrate having a thickness not exceeding 500 micrometers, and having a surface; and
a plurality of non-identical probes immobilized on discrete areas of a probe-containing portion of the substrate surface, each of said discrete areas containing one probe.

21. The apparatus of claim 20 wherein the thickness of the tape does not exceed 100 micrometers.

22. The apparatus of claim 20 wherein the thickness of the tape does not exceed 20 micrometers.

23. A probe-carrying fiber apparatus that is configured to bind samples to form sample-probe complexes, said fiber comprising
a flexible fiber substrate having a length and a diameter, wherein the diameter does not exceed 500 micrometers, and having a surface; and
a plurality of non-identical probes immobilized on discrete areas of a probe-containing portion of the substrate surface, each of said discrete areas containing one probe.

24. The probe-carrying fiber of claim 23 wherein the diameter of the fiber does not exceed 200 micrometers.

25. The probe-carrying fiber of claim 23 wherein the diameter of the fiber does not exceed 100 micrometers.

26. The probe-carrying fiber of claim 23 wherein the diameter of the fiber does not exceed 20 micrometers.

27. An apparatus for depositing a plurality of probes on a substrate, comprising:
a reservoir comprising an array of liquid containing wells; and
a plurality of capillaries, wherein the capillaries each have a first end and a second end, said first end of each capillary is connected to a well of the reservoir to allow liquid content of the well to enter the capillary, said second end of each capillary allows the liquid to exit, and said plurality of second ends are arranged in a single-file row and are capable of depositing probes in a line.
